(19) United States
(12) Patent Application Publication
(10) Pub. No.: US 2024/0412841 A1
(43) Pub. Date: Dec. 12, 2024

(54) A PATIENT SUPPORT PLATFORM FOR INCREASING PATIENT ENGAGEMENT

(71) Applicant: SidekickHealth ehf., Kopavogur (IS)

(72) Inventors: Ólafur Þröstur VIGGÓSSON, Kópavogur (IS); Tryggvi ÞORGEIRSSON, Seltjarnarnes (IS)

(73) Assignee: SidekickHealth ehf., Kopavogur (IS)

(21) Appl. No.: 18/694,400

(22) PCT Filed: Sep. 22, 2022
(86) PCT No.: PCT/IB2022/000543

§ 371 Date: Mar. 21, 2024

Related U.S. Application Data
(60) Provisional application No. 63/394,922, filed on Aug. 3, 2022.
(60) Provisional application No. 63/261,508, filed on Sep. 22, 2021.

(51) Int. Cl. G16H 20/30; G16H 20/60

(52) U.S. Cl. CPC G16H 20/30; G16H 20/60

(57) ABSTRACT

Treatment support systems and methods can select interactive opportunities for presentation to a patient. A system can generate first lists by independently selecting opportunities from an opportunity pool using first models based on competency levels of a patient and associations between the opportunities and competencies of the patient. The system can generate a second list by selecting opportunities from the first lists using a second model based on the competency levels and target competency levels for the patient. The system can provide the second list to a client device of the patient and receive from the client device a selection of an opportunity in the second list. The system can provide the selected opportunity to the client device and receive performance data from the client device. The performance data can concern performance of the selected opportunity. The system can update the competency levels based on the received performance data.

### CROSS REFERENCE TO RELATED APPLICATIONS

This application claims the benefit of U.S. Provisional Application No. 63/394,922, filed Aug. 3, 2022. This application claims the benefit of U.S. Provisional Application No. 63/261,508, filed Sep. 22, 2021. Each of these applications is incorporated herein by reference in its entirety.

### BACKGROUND

Patient support platforms have great potential to improve patient mental and physical health. But when a patient support platform fails to engage a patient, this potential remains unrealized.

Patient support platforms can improve patient engagement by providing relevant, personalized content. But providing relevant, personalized content at scale may present technical challenges. Furthermore, some patients may access patient support platforms through devices with small displays and limited input options. Such patients may find navigating an extensive selection of content cumbersome. Some patients may benefit from immediate feedback on their actions, but providing such feedback through a patient support platform may present technical challenges. Additionally, patients may benefit from frequent interactions with a patient support platform, but encouraging such interactions can be difficult.

### SUMMARY

The disclosed systems and methods relate to a patient support platform configured to provide timely and relevant interaction opportunities to patients.

The disclosed embodiments include a treatment support system. The system can include at least one processor and at least one computer-readable medium containing instructions. When executed by the at least one processor, the instructions can cause the treatment support system to perform operations. The operations can include providing a first mission to a client associated with a user, the user associated with a first treatment pathway. The operations can further include receiving mission data from the client, the mission data concerning performance of the first mission by the client. Based on the received mission data, the treatment support system can associate the user with a second treatment pathway. The operations can further include selecting second missions for provision to the user based, in part, on the association of the user with the second treatment pathway. The operations can include providing an indication of the second missions to the client.

The disclosed embodiments include a treatment support method. The treatment support method can include an associating a user with a first module on a first treatment pathway. The method can further include selecting, at least in part from among missions associated with the first module on the first treatment pathway, first missions for the user to perform. The method can further include providing indications of the first missions to the client and receiving a selection of one of the first missions from the client. The method can further include providing the selected mission to the client and receiving mission data for the selected mission from the client. The method can include automatically determining that the user should be transferred to a second treatment pathway, based on the mission data. The method can further include associating the user with a first module on the second treatment pathway. The method can further include selecting, at least in part from among missions associated with the first module on the second treatment pathway, second missions for the user to perform, and providing indications of the second missions to the client.

The disclosed embodiments include an additional treatment support method. This treatment support method can include selecting, based on a position of a user at a first module on a first predetermined treatment pathway, among first missions associated with the first module. The method can further include providing at least one of the selected first missions to a client for the user to perform, and obtaining, from the client, first mission data concerning performance of the at least one of the selected first missions. The method can include updating the position of the user, based on the obtained first mission data, to a first module on a second predetermined treatment pathway. The method can further include selecting among second missions associated with the first module on the second predetermined treatment pathway. The method can further include providing at least one of the selected second missions to the client.

The disclosed embodiments include an additional treatment support method. This treatment support method can include associating a user with a first module on a first treatment pathway. This method can further include providing missions to a client for the user to perform based on the first module on the first treatment pathway and receiving mission results from the client. The method can include associating, based on the mission results, the user with a first module on a second treatment pathway. The method can include providing missions to the client for the user to perform based on the first module on the second treatment pathway.

The disclosed embodiments include an additional treatment support method for providing personalize treatment support using predetermined treatment pathways. The method can include positioning a patient at a first location, the first location being a first module on a first treatment pathway. The method can further include selecting first treatment support missions for the patient based on the first location. The method can further include providing at least one of the selected first treatment support missions to the patient and receiving mission data for the at least one of the selected treatment support missions. The method can further include identifying, based on the received mission data, a secondary condition of the patient. The method can further include repositioning the patient at a second location, the second location being a first module on a second treatment pathway and providing second treatment support missions for the patient based on the second location.

The disclosed embodiments include a treatment support system. The system can include at least one processor and at least one computer-readable medium containing instructions. When executed by the at least one processor, the instructions can cause the treatment support system to perform operations. The operations can include providing a patient-specific list including a first interactive opportunity to a patient system associated with a patient. The operations can include obtaining performance data from the patient system, the performance data concerning performance of the first interactive opportunity by the patient. The operations can include updating a patient profile of the patient based on the obtained performance data. The operations can include generating a first competency-specific list by selecting among interactive opportunities according to a competency model and the patient profile. The operations can include generating a second patient-specific list of interactive opportunities by selecting among interactive opportunities included in the first competency-specific list and in at least one second competency-specific list according to a care model and the patient profile. The operations can include providing the second patient-specific list to the patient system.

The disclosed embodiments include another treatment support system. The system can include at least one processor and at least one computer-readable medium containing instructions. When executed by the at least one processor, the instructions can cause the treatment support system to perform operations. The operations can include generating first lists by independently selecting interactive opportunities from a pool of interactive opportunities using first models. The first models can select the interactive opportunities from the pool based on competency levels of a patient and associations between the interactive opportunities and competencies of the patient. The operations can include generating a second list by selecting interactive opportunities from the first lists using a second model. The second model can select the interactive opportunities from the first lists based on the competency levels of the patient and target competency levels for the patient. The operations can include providing the second list to a client device of the patient. The operations can include receiving from the client device a selection of an interactive opportunity in the second list. The operations can include providing the selected interactive opportunity to the client device. The operations can include receiving performance data from the client device. The performance data can concern performance of the selected interactive opportunity by the patient. The operations can include updating the competency levels of the patient based on the received performance data.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1 depicts an exemplary architecture for providing a patient support platform, in accordance with disclosed embodiments.

FIG. 2 depicts exemplary contents of a data storage of the architecture of FIG. 1, in accordance with disclosed embodiments.

FIG. 3A depicts an exemplary patient progression architecture based on flexible progression through modules in linked treatment pathways, in accordance with disclosed embodiments.

FIG. 3B depicts an exemplary patient progression architecture based on flexible selection of missions within modules, in accordance with disclosed embodiments.

FIG. 3C depicts an exemplary method of mastery-based progression through the patient architectures depicted in FIGS. 3A and 3B, in accordance with disclosed embodiments.

FIG. 4 depicts a view of an exemplary user interface for providing personalized treatment support, in accordance with disclosed embodiments.

FIG. 5A depicts interactive opportunity completion views of an exemplary user interface for providing personalized treatment support, in accordance with disclosed embodiments.

FIG. 5B depicts an interactive opportunity customization view of an exemplary user interface for providing personalized treatment support, in accordance with disclosed embodiments.

FIG. 6 depicts a pathway information view of an exemplary user interface for providing personalized treatment support, in accordance with disclosed embodiments.

FIG. 7 depicts a module information view of an exemplary user interface for providing personalized treatment support, in accordance with disclosed embodiments.

FIG. 8 depicts an exemplary game loop for an application for providing personalized treatment support, in accordance with disclosed embodiments.

FIG. 9 depicts a daily progress view and a completion view of an exemplary user interface for providing personalized treatment support, in accordance with disclosed embodiments.

FIG. 10 depicts views associated with opening a prize, in accordance with disclosed embodiments.

FIG. 11A depicts a view of a coach portal interface for providing personalized treatment support, in accordance with disclosed embodiments.

FIG. 11B depicts a bulk messaging window for communicating with multiple patients, in accordance with disclosed embodiments.

FIG. 11C depicts a patient information view, in accordance with disclosed embodiments.

FIG. 11D depicts a message window for communications between a coach and a patient, in accordance with disclosed embodiments.

FIG. 12 depicts a patient-reported outcome (PRO) feedback view of a coach portal interface for providing feedback on patient reported outcomes, in accordance with disclosed embodiments.

FIG. 13 depicts a performance view of a coach portal interface for reviewing user mission performance, in accordance with disclosed embodiments.

FIG. 14A depicts a food journal view of a coach portal user interface for reviewing user food journals and providing feedback, in accordance with disclosure embodiments.

FIG. 14B depicts a food entry view of a coach portal user interface for reviewing an individual food journal submission, in accordance with disclosed embodiments.

FIG. 14C depicts a patient view of a patient user interface, in accordance with disclosed embodiments.

FIG. 15 depicts exemplary contents of a second data storage of the architecture of FIG. 1, in accordance with disclosed embodiments.

FIG. 16A depicts an exemplary architecture for selecting personalized interactive opportunities, in accordance with disclosed embodiments.

FIG. 16B depicts exemplary inputs to the care model used to select personalized interactive opportunities, in accordance with disclosed embodiments.

FIG. 16C depicts an exemplary method for selecting personalized interactive opportunities, in accordance with disclosed embodiments.

### DETAILED DESCRIPTION

Reference will now be made in detail to exemplary embodiments, discussed with regard to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Patient support platforms have great potential to improve patient mental and physical health. Such platforms can educate patients about their conditions, provide a forum for social interactions and support, and promote beneficial behavior modifications. But when a patient support platform fails to engage a patient, this potential remains unrealized. A patient that does not access a platform cannot be educated, socially supported, or trained through that platform.

Patient support platforms can improve patient engagement by providing relevant, personalized content. But such content can be difficult to provide at scale, as different patients have different needs. For example, a first patient may require assistance with medical management, while a second patient might require assistance with pain management or strength training. Providing the second patient content concerning medication management could cause them to disengage from the platform. Different patients also progress towards treatment goals at different rates. For example, the first patient may rapidly adopt a healthier diet in response to prompts provided through a patient support platform, while the second patient may continue to struggle. Content appropriate for the second patient may appear patronizing or irrelevant to the first patient and could cause them to disengage from the platform. Configuring a platform to provide relevant, personalized content upon patient enrollment can also be difficult, as patients may be unaware of their own needs or unwilling to participate in an extensive onboarding process. For at least these reasons, existing patient support platforms may struggle to provide relevant, personalized content.

Patient engagement can be limited by characteristics of the devices that patients use to interact with patient support platforms. Patients often interact with patient support platforms through devices, such as mobile or wearable devices, that have small displays and limited input options. Small displays may increase the importance of selecting the most timely and relevant content, while limited input options may make communication with clinicians more difficult.

Patients may interact with patient support platforms frequently, and in causal settings. Patient expectations for such interactions may differ from occasional, more-formal interactions, such as consultations with a doctor or dietician at a clinic. For example, as compared to in-person interactions between a patient and clinician, interactions through a patient support platform can appear impersonal, distant, or alienating. Furthermore, in-person interactions may allow for immediate feedback, while feedback through a patient support platform can be delayed (and such delayed feedback can become more irritating the more frequently the patient accesses the platform).

Patient support platforms may provide the most benefit when patients access them frequently. But treatment goals may appear distant or unachievable, while progress in realizing treatment goals may be difficult to assess. Accordingly, a patient may lack a sense of control or investment in their treatment, which may cause the patient to access the patient support portal infrequently, reducing the effectiveness of the platforms.

The disclosed embodiments provide technical solutions to these technical problems with existing patient support platforms. The disclosed embodiments provide relevant, personalized content through an architecture based on scalable personalization. Patient onboarding can be expedited using patient medical information and/or predicting patient preferences. Furthermore, a patient support platform consistent with disclosed embodiments can be architectured to increase patient engagement though interface design, built-in support for coaching and patient interaction, automatically generated feedback for patient actions, and gamification. In this manner, the disclosed embodiments can provide technical improvements over existing patent support platforms.

The envisioned embodiments achieve scalable personalization using linked treatment pathways, provision of multiple interaction opportunities, and/or configuration of individual interaction opportunities. Treatment pathways and interaction opportunities can be created in advance and provided to multiple patients. Accordingly, a patient's interactions with the patient support platform can determine the particular interaction opportunities offered to that patient. By permitting customization at multiple levels, this architecture enables creation of complex, patient-driven treatments from pre-existing constructs (e.g., treatment pathways, missions, or the like). The envisioned architecture therefore increases the scalability of the system, while simultaneously supporting patient engagement by providing relevant, personalized content.

In some embodiments, the patient support platform can support personalization through linkable treatment pathways. A treatment pathway can be a structured arrangement of content (and can be represented, for ease of discuss, as a directed graph). A treatment pathway can be arranged to guide a patient with a particular condition (e.g., rheumatoid arthritis, or the like) towards a particular primary clinical endpoint (and optionally one or more secondary clinical endpoints). A treatment pathway can be developed in consultation domain experts and validated by relevant stakeholders (e.g., insurance providers, care providers, governmental agencies, or the like) prior to deployment. A treatment pathway can include decision points linking the treatment pathway with other treatment pathways. Under certain predetermined conditions, a patient can be transferred to another treatment pathway (or the other treatment pathway can be associated with the patient). For example, a patient can be assigned to an initial, primary care pathway. Progression along this primary care pathway can be mastery-based, depending on the development of a competency (e.g., a skill, attribute, or characteristic of the patient, such as sleep management, pain management, medication adherence, physical activity level, depression, weight management, or the like). For example, the patient support platform can determine that a patient satisfies a progression requirement for a competency and assign the patient to the next position (e.g., module, or the like) along the pathway. As an additional example, the patient support platform can determine that the patient does not satisfy a progression requirement for the competency and transfer the patient to a treatment pathway designed to develop that competency. For example, the physical activity level of the patient can be a competency. Progression along the initial, primary treatment pathway may require sufficient development of this competency. A secondary treatment pathway can be structured to increase the physical activity level of the patient. For example, the secondary treatment pathway can include more interaction opportunities that concern physical activity than the primary treatment pathway. If the patient has not demonstrated a sufficient physical activity level, they may be transferred from the primary treatment pathway to the secondary treatment pathway.

In some embodiments, the patient support platform can support personalization through selection of interaction opportunities. The patient support platform can select these interaction opportunities based on an association between the patient and a treatment pathway. The patient support platform can select the interaction opportunities further based on an association between the treatment pathway and the interaction opportunities. For example, when selecting interaction opportunities to provide to a patient, the patient support platform can select among interaction opportunities included in or referenced by treatment pathways associated with the patient. Such interaction opportunities can include missions, as described herein, educational content, reporting opportunities, or the like. The selection of interaction opportunities can depend on prior patient behavior. For example, when different types of interactive opportunities are substitutable, the patient support platform can include more patient-preferred interactive opportunities. In this manner, the patient can receive a personalized selection of interactive opportunities.

As may be appreciated, while interactions are described as being between the patient and the platform, such interactions can also be between the platform and a “representative of the patient (e.g., parent, child, spouse, guardian, clinician, or other person responsible for interacting with the patient support platform on behalf of the patient).

In some embodiments, the patient support platform can provide scalable personalization by offering patients customizable interaction opportunities. Once a patient has selected an offered interaction opportunity, the patient support platform can provide options for customizing the selected interaction opportunity. In some instances, a mission can have parameters that affect the characteristics of the mission. For example, a physical activity mission might offer a choice of physical activities, a choice of the duration or intensity of a physical activity, or other, similar choices. Similarly, a mental mission may offer a selection of meditative activities, or an educational mission may permit the patient to select among educational materials or provide an optional quiz. The customization opportunities can depend upon prior patient behavior. For example, a physical mission could require the patient to walk for a certain duration. The patient support platform could offer the patient the opportunity to provide this duration, and/or could preselect a duration based on the patient's past performance of similar missions. The platform could obtain the patient's past performance from the patient (e.g., the patient could enter a duration walked in a prior physical mission), from a wearable device of the patient, from a clinical or coach of the patient, from a medical record of the patient, or the like. In this manner, the interactive opportunities selected by the patient can be further personalized.

In some embodiments, the patient support platform can use mastery-based progression to increase the relevance of interactive content provided to the patient. As described herein, treatment pathways can be structured as a sequence of modules. The patient can progress through the treatment pathway from module to module. Progression to the next module can require the patient satisfy a competency criterion. For example, the patient support platform can prevent a patient from progressing beyond an initial module in a rheumatoid arthritis treatment pathway until the patient can demonstrate knowledge of the symptoms, causes, and treatments of rheumatoid arthritis. Because progression to new modules (and therefore new missions) is keyed to patient competencies, the patient support system can ensure that patients are provided with missions of appropriate difficulty. For example, a patient unable to walk would not be provided a physical mission that requires jogging for 5 minutes. Furthermore, as opposed to alternative embodiments that use a time-based progression system (e.g., one in which a patient progresses to the next module at a predetermined time), a patient that does not log into the platform regularly does not fall behind. In this manner, the envisioned mastery-based progression can support increased patient engagement with the platform.

In some embodiments, the patient support platform can reduce the burden of patient onboarding using obtained patient medical information (e.g., patient diagnoses, disorders, diseases, or conditions; weight, blood pressure, age, or other health data; medications, surgical interventions, or other treatments; or similar medical information). The medical information can be obtained from a medical record associated with the patient, or by scanning a prescription, medication container, or medication (e.g., a pill or the like). For example, the patient support platform can instruct the patient to take a photo of a medication bottle relevant to the patient's condition using a smartphone. The photo can be provided to the patient support platform, which can use optical character recognition, barcode or QR code reading, or the like, to identify the medication. In some embodiments, the patient support platform can use the obtained medical information to select the initial interactive content presented to the patient. For example, the patient can be associated with a primary treatment pathway based on the obtained medical information. The initial interactive content presented to the patient can then be based on the primary treatment pathway. For example, if a medical record associated with the patient indicates that the patient is overweight, the patient support platform can be configured to including interactive content concerning the benefits of weight loss, or missions concerning healthy eating, or the like, among the initial interactive content.

In some embodiments, the patient support platform can reduce the burden of patient onboarding by predicting patient configuration preferences. Such configuration preferences can include the primary treatment pathway associated with the patient, the mix of interactive opportunities provided to the patient, and configuration options of individual interactive opportunities. The patient support platform can predict patient configuration preferences for new patients based on the configuration preferences of existing patients. The prediction can depend on patient medical information, demographic information, location, or the like, for the new patient and for the existing patients. The patient support platform can use a nearest neighbor algorithm, clustering algorithm, neural network, decision tree or random forest, or similar statistical or machine learning model (or combination thereof) to predict a configuration of the patient support platform for a new patient.

In some embodiments, the patient support platform can support increased patient engagement through an improved user interface that presents patient information using a hierarchy of views. The patient can traverse this hierarchy to obtain information concerning a current treatment pathway (e.g., among multiple pathways associated with the patient), a current module within a treatment pathway, currently available interactive opportunities, or a particular interactive opportunity. Even when using a device with limited interactive options, such as a smartphone, a patient can interact with envisioned interfaces to quickly transition from high-level information (e.g., their location in a treatment pathway) to specific information (e.g., the completion requirements of a particular mission). In this manner, the envisioned patient support platform user interfaces can improve useability and support increased user engagement.

In some embodiments, the patient support platform can provide relevant content by selecting interaction opportunities based on the location of the patient within a set of linked treatment pathways. For example, a patient may initially be placed on a primary treatment pathway, then transferred to a secondary treatment pathway to develop a particular competency. In some instances, the secondary treatment pathway may supersede the primary treatment pathway: the patient support platform may only select interaction opportunities for display to the patient from among those associated with the secondary treatment pathway. In various instances, the secondary treatment pathway may complement the primary treatment pathway: the patient support platform may select interaction opportunities for display to the patient from among those associated with both the primary and the secondary treatment pathway. To avoid overwhelming a patient with choices, the patient support platform can be configured to select a subset of possible interaction opportunities. This subset can be selected according to an optimization process. For example, the patient support platform can select interaction opportunities based on weights associated with each interaction opportunity (e.g., using a bin-packing algorithm, or the like). The weights can be dynamic (e.g., time-varying or updated in response to patient actions), and can depend on the interaction opportunity, the treatment path referencing or containing the interaction opportunity, and the patient's prior interaction with the platform. In this manner, the patient support platform can increase the relevance of the interaction opportunities displayed to the patient, thereby supporting increased patient engagement.

In some embodiments, the patient support platform user interface can support increased patient engagement through built-in coaching functionality. Such functionality can address the lack of personal contact that afflicts existing patient support platforms. Consistent with disclosed embodiments, the patient support platform user interface can include a coaching portal that enables coaches to interact with one or more patients. The coaching portal can be web-based, enabling coaching by a practitioner treating the patient or affiliated with the patient's health-care provider (e.g., third-party coaching) or a practitioner affiliated with the entity providing the patient support platform (e.g., second-party coaching). The coaching portal can support improved scalability by enabling a coach to simultaneously (e.g., in a single view) review the statuses and actions of multiple patients.

In some embodiments, the patient support platform can limit disclosure of patient information (e.g., patient medical information, demographic information, location information, or the like) to coaches. Such information can be limited by platform administrators for coaches generally, for specific coaches, for patients generally, for specific patients, or any combination of the forgoing. For example, the patient support platform can be configured to permit coaches in general to know both what medication a patient is taking and when they last reported taking that medication. But the patient support platform can be reconfigured to limit this information for a particular coach (or for a particular patient). For this particular coach (or with regards to this particular user), the patient support platform may limit disclosure to when the patient last reported taking a medication (and omit what medication the patient is taking). Such limitations can be imposed on a geographic basis (e.g., information about patients in Europe may be more limited that information about patients elsewhere).

In some embodiments, the coach portal can permit assignment of a patient to multiple coaches. Each of these coaches can handle an aspect of the patient's experience. For example, a nutritionist can provide feedback to patients regarding healthy eating choices, a psychiatrist can provide feedback regarding patient mental health, and a nurse practitioner can provide feedback regarding pain management. Each coach can interact with the patient through a coaching portal that only presents patient information relevant to that coaches' role. For example, the coach portal accessed by the nutritionist may display only diet-related information, while the coach portal accessed by the psychiatrist may not display diet-related information. While different coaches may interact with the patient through different coaching portals, the patient may interact with the coaches through a single interface. In this manner, the patient support platform can enable a beneficial division of labor, with coaches specializing on particular areas, while still presenting the patient with a unified coaching experience.

In some embodiments, the coach portal can support interactions between coaches and patients. The interactions can be designed to further treatment and prevent patient disengagement. For example, patients can send messages or initiate chat sessions with coaches. These interactions can address problems (e.g., medication issues, health or treatment concerns, technical issues with the patient support platform, or the like) that might otherwise inhibit treatment, while providing a sense of connection that can reduce the risk of a patient disengaging from the platform. As an additional example, coaches can receive messages automatically generated by the platform concerning patients. Such messages can be triggered by patient actions (e.g., competition of a mission or set of missions, progression along a treatment path, or the like) or inactions (e.g., not logging into the platform for an amount of time, not completing missions, not selecting missions, or the like). As a further example, coaches can provide messages to patients (e.g., feedback on patient performance), schedule in-person consultations, or engage in chat or teleconference sessions. In some embodiments, a coach can modify the status of a patient within the program. For example, the coach may be able to transfer a patient to another treatment pathway, or to another module within a treatment pathway.

In some embodiments, the patient support platform can be configured to automatically generate feedback in response to patient actions. Delayed feedback can irritate patients or cause them to feel abandoned or ignored. Automatically generated feedback can supplement feedback provided by coaches, preventing patients from experiencing delayed feedback. In this manner, automatically generated feedback can improve the patient experience and support greater patient engagement with the patient support platform.

In some embodiments, the patient support platform can use gamification techniques to improve patient engagement with the patient support platform. Such gamification techniques can include creation of a core gameplay loop. In such a gameplay loop, a patient can be rewarded for interactions with the platform (e.g., completion of interactive opportunities, advancement along a treatment pathway, responding to feedback from coaches, or other interactions). In various embodiments, rewards can include additional customization or personalization of the patient's experience with the platform, status enhancements within the community of patients using the platform, or non-virtual rewards to the patient or a third party. The gamification techniques can include bonuses and special offers, progress indicators, gameplay modifiers, and the like, designed to increase patient engagement. In addition, the interface can support forums, rankings, and other community-building social elements. In this manner, the patient support platform can provide patients a sense of control or investment in their treatment, improving patient engagement with the platform.

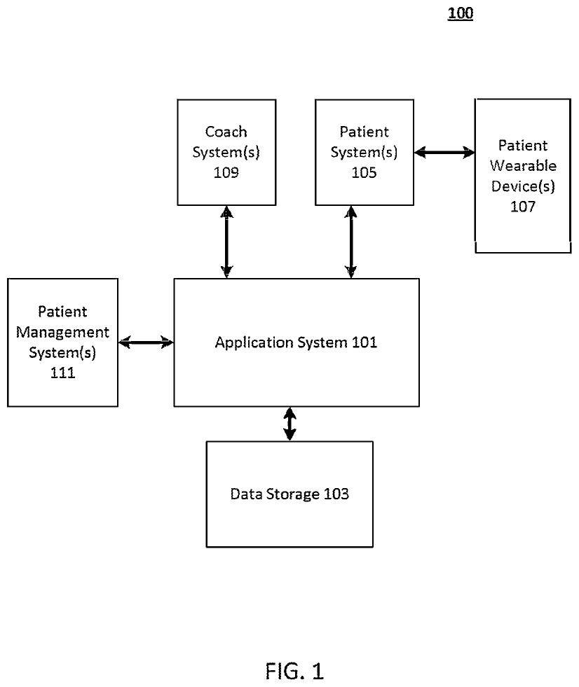

FIG. 1 depicts an exemplary architecture 100 for providing a patient support platform, in accordance with disclosed embodiments. As described herein, architecture 100 can support provision of timely, relevant interaction opportunities to patients, thereby improving patient engagement. Improved patient engagement can in turn improve the therapeutic benefits of the platform for patients.

Architecture 100 can include application system 101 and data storage 103. Applicant system 101 and data storage 103 can be implemented using at least one computing system. In some embodiments, application system 101 and/or data system 103 can be implemented using a cloud computing system (e.g., Google Could, Amazon AWS, or the like).

In some embodiments, application system 101 can by implemented as a collection of containerized services. The containerized services can expose endpoints for responding to client requests (e.g., representational state transfer endpoints implemented in Java, or the like). Deployment and management of these services can be achieved using a container orchestration system (e.g., Kubernetes, Docker, Nomad, or the like). In some embodiments, the containers can include, or be configured to connect to volumes that include, the data and instructions that specify the patient support platform (e.g., platform components 210).

In some embodiments, data storage 103 can be implemented using, in part, an SQL database (E.g., Google Cloud SQL, or the like). The SQL database may be configured to store account information for patients (e.g., patient profile information 220, as described herein). In some embodiments, the SQL database may periodically be de-identified and backed up to a data warehouse (e.g., Google BigQuery, or the like). In some embodiments, data storage 103 can be further implemented using, in part, general cloud storage (E.g., Google Cloud Storage, or the like). Such general storage may include storage of files or attachments associated with communications between users of architecture 100.

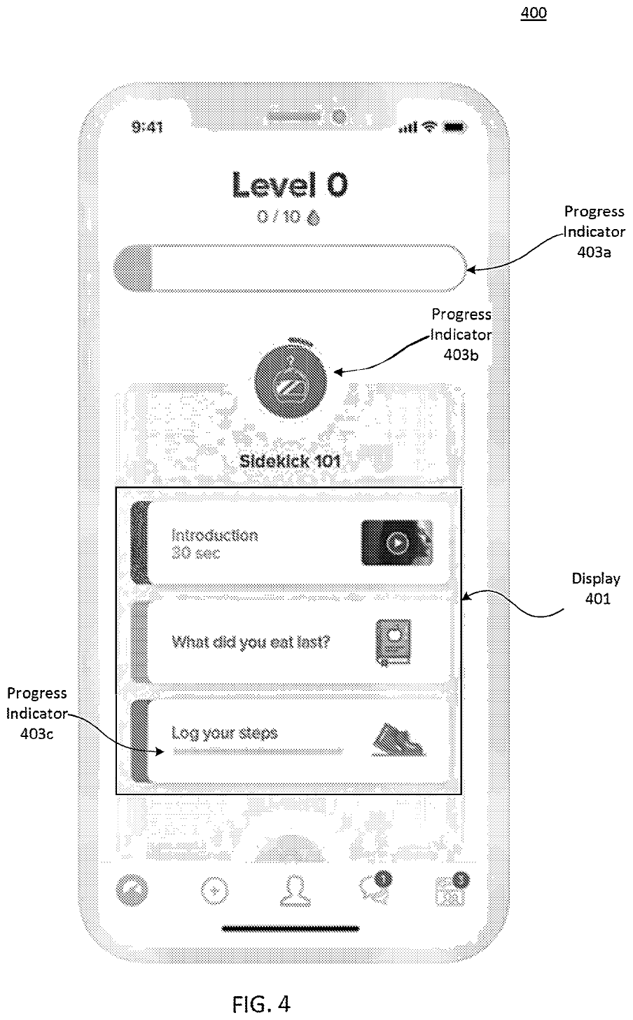

Architecture 100 can include patient system(s) 105, consistent with disclosed embodiments. Such patient systems can include mobile computing devices (E.g., smartphones, tablets, laptops, or the like), or other computing devices (e.g., desktop computers or the like). In some embodiments, the patient systems can be configured to interact with applications system 101 using an application (e.g., a client application native to the particular operating system of the patient system). The application can provide an engaging user interface, as described herein with regards to FIGS. 4 to 10, and handle communications between the patient system and the application system 101. In some embodiments, in addition or as an alternative to using the application, patients can access application system 101 through a web portal. In some embodiments, patient health system(s) 105 can provide to application system 101 patient health information (e.g., number of steps taken, heart rate, pulse oximetry, EEG data, or the like) obtained from patient wearable device(s) 107. In some embodiments, the application on the patient system can communicate with application system 101 using HTTPS, or a similar protocol.

Architecture 100 can include patient wearable devices(s) 107, consistent with disclosed embodiments. Such wearable devices can include smartwatches (e.g., Apple Watch, Samsung Galaxy Watch, or the like), fitness trackers (e.g., Fitbit, or the like), headsets (e.g., MUSE, NeoRythm, or the like), or other similar wearable devices. In some embodiments, as shown in FIG. 1, a patient wearable device can communicate with a patient system, enabling the patient system to obtain data regarding the patient's health. In various embodiments, the wearable device may be configured to provide this patient health data to a remote system (e.g., an application server associated with the provider of the wearable device). The patient system can be configured to obtain the patient health data from the remote system. In some embodiments, the wearable device may be configured to provide the patient health data directly to application system 101.

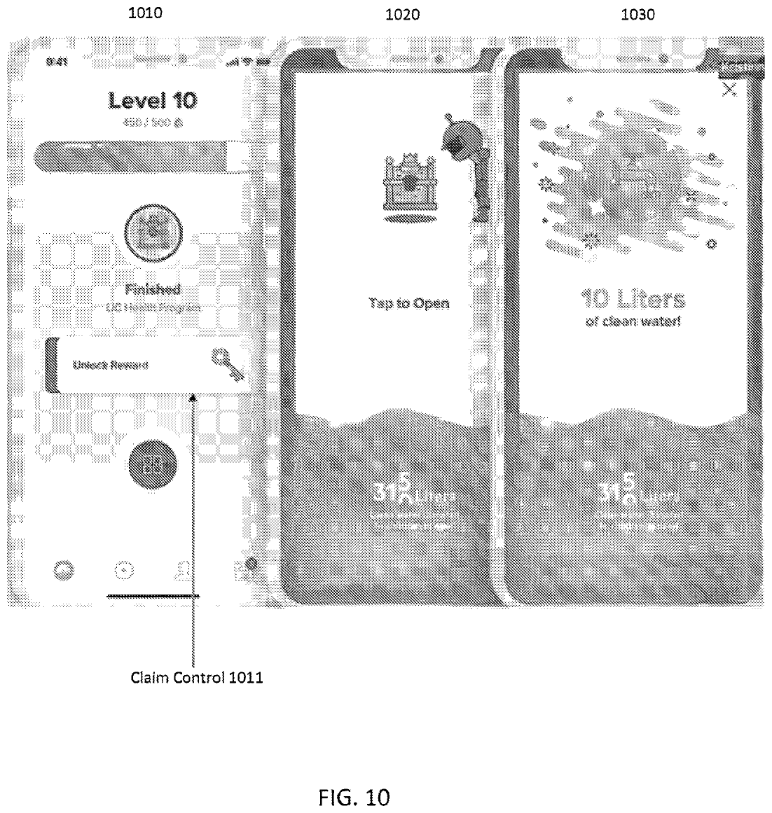

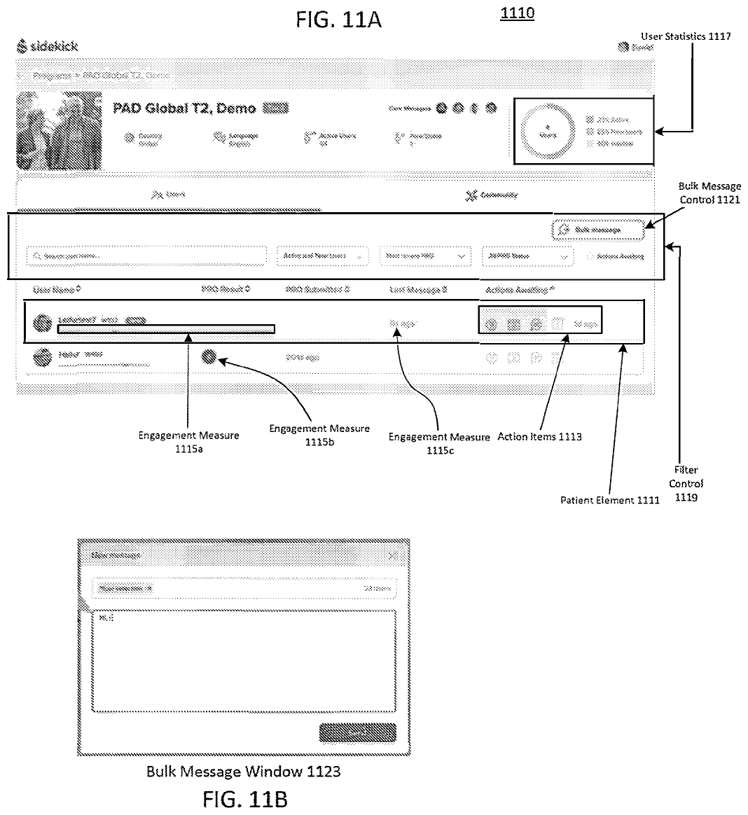

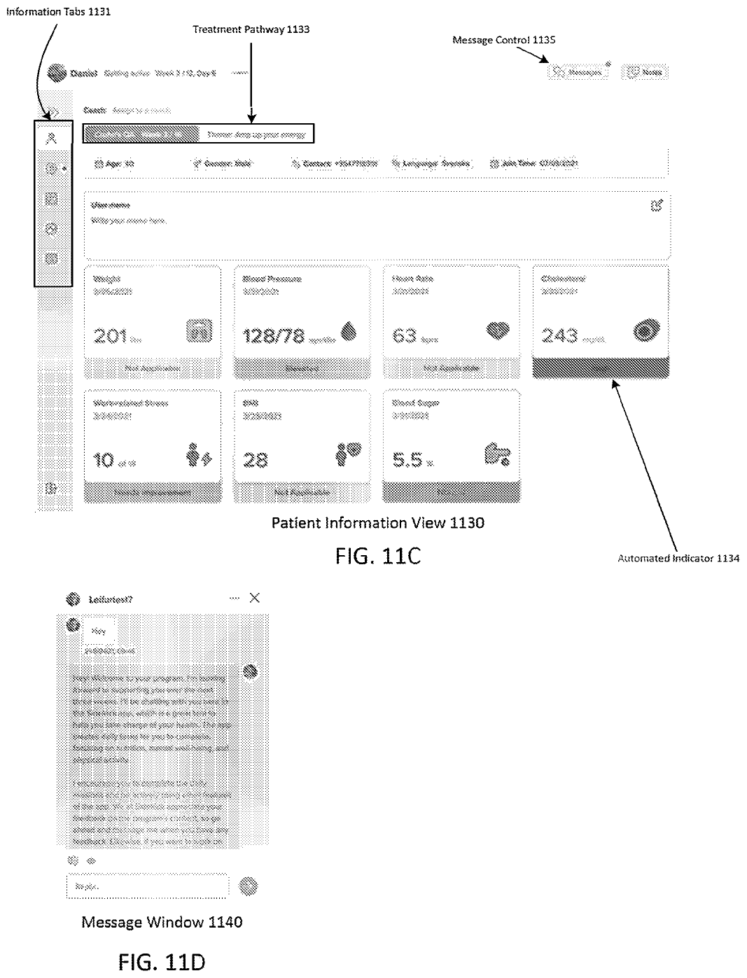

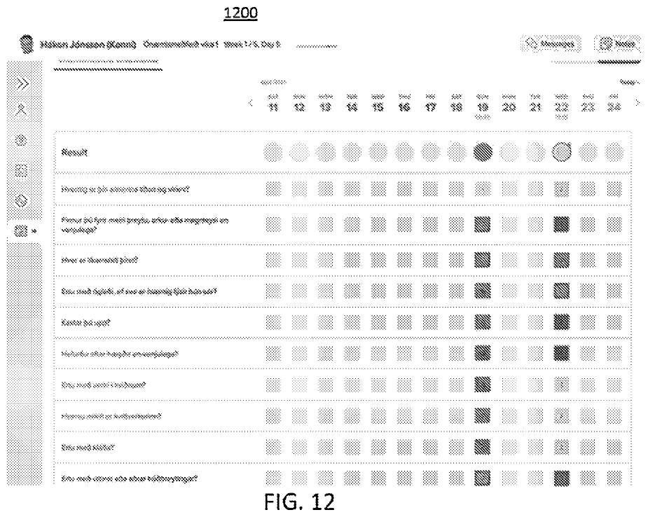

Architecture 100 can include coach system(s) 109, consistent with disclosed embodiments. A coach system can be a computing device (e.g., a mobile device, such as a smartphone, tablet, laptop, or the like; or a desktop, terminal, workstation, or the like). A coach can use a coach system to access application system 101 through a web portal, as described herein with regards to FIGS. 11A to 14C.

Architecture 100 can include patient management system(s) 111, consistent with disclosed embodiments. A patient management system can be configured to maintain patient electronic medical records. The patient management system can be controlled by an entity (e.g., a hospital network, an insurance company, ort the like) distinct from the entity controlling application system 101. In some embodiments, there can be a one-direction linkage between the electronic medical records maintained by patient management system(s) 111 and application system 101: changes made to the electronic medical records can flow to application system 101. In various embodiments, there can be a two-direction linkage between the electronic medical records maintained by patient management system(s) 111 and application system 101: the changes in one system can flow to the other. Consistent with disclosed embodiments, patient management system(s) 111 can provide application system 101 with patient health information. Application system 101 can use this information to recommend interactive opportunities to the patient as described herein.

Though described for convenience as a system in which the client application handles displaying interactive opportunities and relaying patient inputs back to application system 101, the disclosed embodiments are not limited to any particular division of functionality between the application system 101 and the client application. Interactive opportunities can be stored on application system 101 or the client system. The status of the patient (e.g., treatment pathways associated with the patient, modules assigned to the patient, history of patient interactions, or the like) can be stored on application system 101 or the client system. Application system 101 or the client application can select interactive opportunities for display to the patient. In some embodiments, the client application can handle maintenance of patient status and selection, display, and configuration of interactive opportunities. In such embodiments, the client application can provide to application system 101 indications of which missions the patient selected, which missions the patient completed, and any communications intended for the coaches.

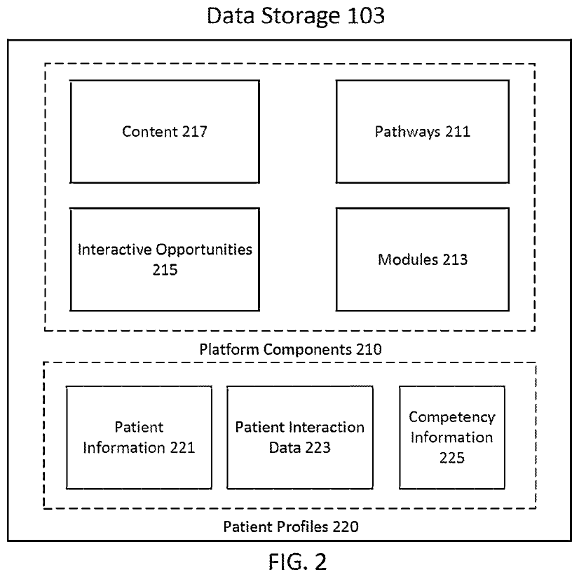

FIG. 2 depicts exemplary contents of data storage 103, in accordance with disclosed embodiments. The depiction in FIG. 2 is a logical depiction: the depicted contents of data storage 103 can be distributed across multiple physical machines (including, in some embodiments, patients' system(s) 105). The depicted contents can include platform components 210 and user profiles 220. Platform components 210 can include objects or data useable with multiple patients, while patient profiles 220 can include objects or data associated with a particular patient. The disclosed embodiments are not limited to any particular implementation or format for platform components 210 or patient profiles 220.

Platform components 210 can include pathways 211, modules 213, interactive opportunities 215, and content 217. Pathways 211 can include data or instructions that implement treatment pathways accessible to patients through the patient support platform. In some embodiments, a treatment pathway can define a sequence of modules that a patient may traverse over the course of treatment. Modules 213 can include data or instructions that specify a set of interactive opportunities. As described herein, patient can be associated with a module. When selecting interactive opportunities for display to the patient, the patient support platform can select among interactive opportunities specified for the module associated with the patient. Interactive opportunities 215 can include data or instructions for implementing missions, questionnaires, data submissions, patient communications, or the like (e.g., interactive opportunities). In some embodiments, pathways 211, modules 213, and interactive opportunities 215 may include instructions for referencing items of content 217. Content 217 can include interface content used by the patient support platform interface (e.g., icons, interface graphics, sounds, or the like), educational content (e.g., concerning diseases or conditions, treatments, or the like), questionnaires, or the like. In some embodiments, content 217 can include graphical, audio, or audiovisual content, or other suitable content.

Patient profiles 220 can include patient information 221, patient interaction data 223, and competency information 225. Patient information 221 can include patient medical information, demographic information, location information, or the like. Patient information 221 can include information obtained from the patient, information obtained from another party regarding the patient (e.g., a parent or guardian, physician, or employer of the patient, or the like), a medical record of the patient (e.g., a medical record accessed or retrieved from patient management system(s) 111), activity data obtained from a device associated with the patient, or the like. Patient interaction data 223 can include a record of the interactive opportunities selected by the patient, customization options selected by the patient for the selected interactive opportunities, whether the selected interactive opportunities were completed, data pertaining to the performance of the selected interactive opportunities (e.g., steps taken, minutes meditated, questions answered correctly, patient-reported outcome results, patient submitted comment text, or the like), communications between the patient and a coach, amount of time logged into the platform, or other suitable interactions with the patient support platform. Competency information 225 can include data indicating development of a competency (e.g., a skill, attribute, or characteristic of the patient). Such competencies can concern, for example, sleep management (e.g., indicating patient quality of sleep, length of sleep, appropriateness of bedtime or risetime, adherence to a sleep schedule, or the like), pain management, physical activity level (degree of physical exercise, setting of physical exercise goals, or the like), mental state (e.g., depression or mood, energy level, stress or relaxation level, meditation practice, or the like), diet management (e.g., appropriateness of food intake, food journaling, setting and achieving food goals, or the like), hydration management (e.g., appropriateness of water intake), weight management, treatment-engagement level (e.g., medication adherence, knowledge of condition or treatment, performance of patient related outcomes and other interactions, or the like) or the like. Data for a competency (e.g., physical activity level) can indicate a level or categorization (e.g., beginner, intermediate, expert, or the like), or a value (e.g., average number of minutes spent in physical activity in a week, average number of steps in a day, or the like).

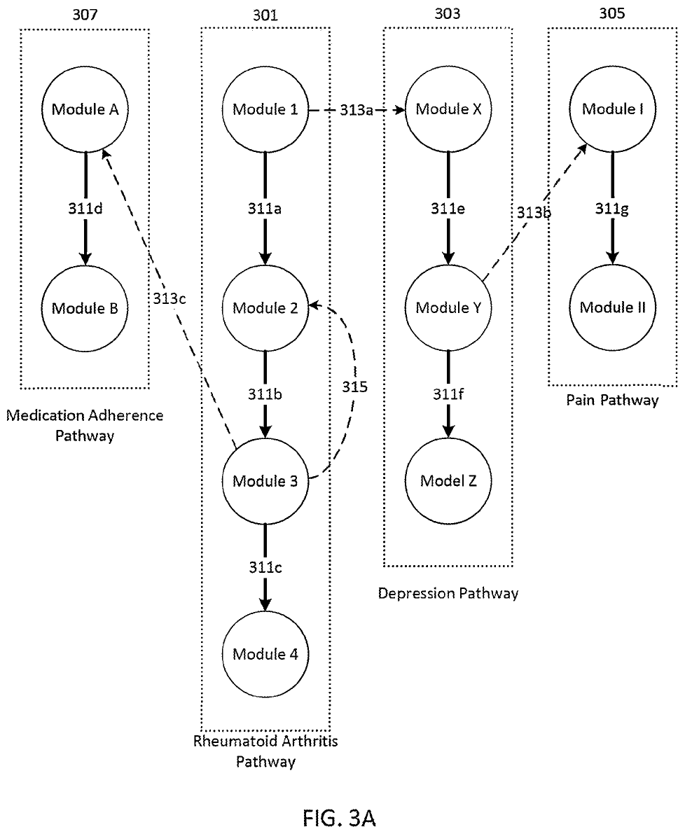

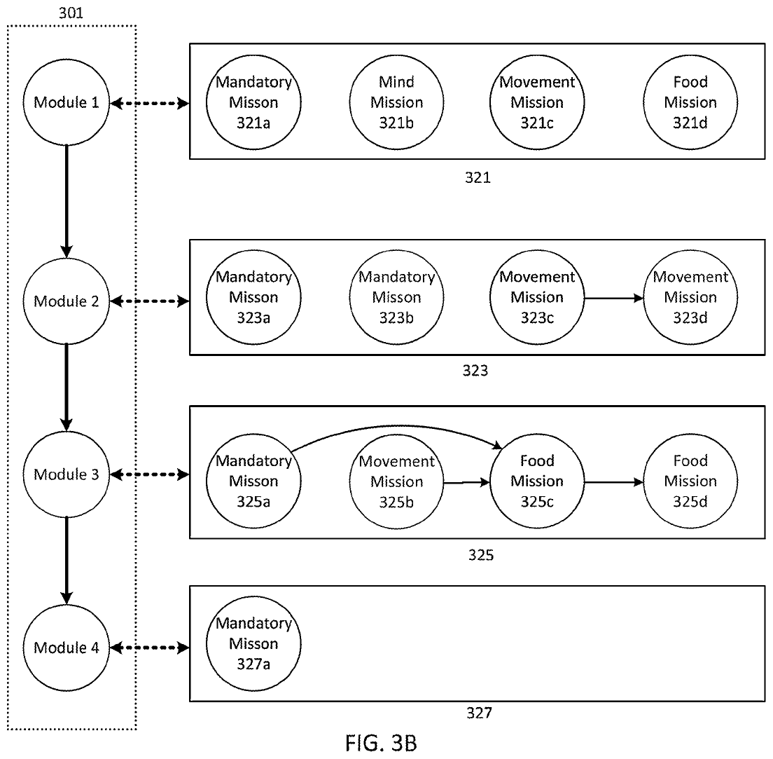

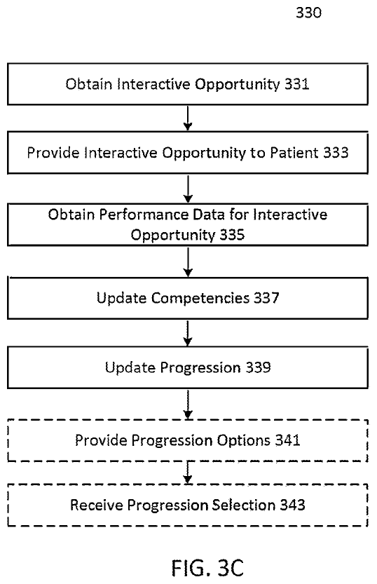

FIG. 3A depicts an exemplary patient progression architecture based on flexible progression through modules in linked treatment pathways, in accordance with disclosed embodiments. For simplicity, the example depicted in FIG. 3A includes four treatment pathways, while envisioned implementations could include tens to hundreds of treatment pathways, or more. A treatment pathway can include modules (e.g., module 1, module Y, module II, module A, as depicted in FIG. 3A). The modules in a pathway can form a sequence, connected by links (e.g., links 311a, 311b, 311c, 311d, 311e, 311f, 311g, or the like). Once a patient satisfies a progression criterion associated with a module, the patient support platform can assign them to the next, linked module in the pathway. As, in some embodiments, the interactive opportunities presented to the patient depend on the module(s) assigned to the patient, the sequential structure of the treatment pathways can support provision of relevant content to patients.

Consistent with disclosed embodiments, the progression criterion can include at least one of a time requirement, a performance requirement, a mastery requirement, a data-dependent requirement, or the like. A time requirement can specify that a patient must remain assigned to a module for at least a certain duration (e.g., the patient can progress to the next module after a week). A performance requirement can specify that the patient complete at least a certain number of interactive opportunities (e.g., completion of ten of twelve missions associated with module 1), or at least a certain number of interactive opportunities in each of at least a certain number of interactive opportunity types (e.g., performance of two physical activity missions, two food mission, two mind missions, and two educational missions). A mastery requirement can depend on patient competencies. For example, a patient may not progress from module 1 of pathway 301 until they have achieved a “beginner” level in the physical activity competency. In some embodiments, the patient support platform can determine or track competencies for the patient based on patient interactions with the platform. In some embodiments, successfully completing interactive opportunities can boost competencies associated with those interactive opportunities. For example, successfully completing physical activity missions can increase a patient's physical competency, while successfully completing education missions can increase a patient's education competency.

Successful completion of an interactive opportunity can be indicated by the patient directly, through interactions with the patient support platform (e.g., selecting a control to indicate completion of a mission, responding to all the questions in a questionnaire, or the like), or indirectly, through data obtained from a wearable device associated with the patient (e.g., electroencephalogram data obtained from a headset, heart-rate or steps-taken data obtained from a smartwatch, or the like). A data-dependent requirement can specify conditions on patient information obtained from a medical record (e.g., patient weight data from a physical examination, or cholesterol data from laboratory bloodwork). Such data can be obtained, for example, from patient management systems 111.

Consistent with disclosed embodiments, a treatment pathway can include link(s) between modules within the pathway (e.g., link 315). Such links can break the ordinary sequence of modules within the treatment pathway and thereby support flexibility in responding to patient progression (or regression). For example, upon completion of module 3, the patient support platform could determine whether the patient has satisfied the conditions for progression to module 4. If not, rather than merely repeating module 3, the patient support platform may return the patient to module 2 (e.g., through link 315), to bolster some necessary competency, receive additional education, or the like. Alternatively, when a patient demonstrates unusually rapid development of competencies, a link could advance them out of sequence (e.g., such a link could advance the patient from module 1 to module 3). When a patient satisfies a reassignment criterion associated with such a link, the patient support platform can reassign them to the linked module in the pathway. Similar to progression criterions, such reassignment criterion can include at least one of a time requirement, a performance requirement, a mastery requirement, a data-dependent requirement, or the like.

Consistent with disclosed embodiments, a treatment pathway can include link(s) to other, destination treatment pathway(s) (e.g., links 313a, 313b, 313c). The treatment pathway can correspond to an original or primary complaint of the patient, while the destination treatment pathway(s) may correspond to potential secondary complaint(s) of the patient. As described herein, such secondary complaint(s) may become apparent as a patient interacts with the patient support platform. When transfer conditions for a link in the originating treatment pathway are satisfied, the patient support platform can transfer a patient from the originating treatment pathway to a destination treatment pathway along the link. Such links can be implemented to allow independent updating of pathways. For example, pathway 301 can implement a connection 313a to pathway 303. Pathway 303 can be updated without breaking this connection.

Consistent with disclosed embodiments, transfer conditions can depend upon patient interactions with the patient support platform, coach input, data received from patient medical records, or the like. Such patient interactions can include completion of a patient report (e.g., a questionnaire, or the like), the results of which satisfy the transfer condition, or the selection (or non-selection, or the like) of certain types of interactive opportunities (e.g., non-selection of physical missions, or the like), or the quality of performance of certain interactive opportunities (e.g., when a mission includes documenting food choices, a failure to select healthy meals). Coach input can include an instruction received by the patient support platform from a coach associated with the patient to transfer the patient. For example, a treatment pathway may provide coaches the opportunity to transfer patients to another pathway. The coach could interact with the patient support platform to transfer the patient. The coach may decide to transfer the patient based on the content of communications between the patient and the coach, a lack of progression by the patient along the treatment pathway, or other suitable criteria.

Consistent with disclosed embodiments, the patient support platform can select interactive opportunities for provision to the patient. This selection can depend on the module(s) and pathway(s) associated with the patient. In some embodiments, the patient support platform can consider all the module(s) assigned to the patient. In various embodiments, the patient support platform can consider a subset of these module(s). In such embodiments, the patient support platform can consider the module(s) included in a subset of the treatment pathways associated with the patient. In some embodiments, the subset of the treatment pathways can include the primary treatment pathway. In various embodiments, the subset of the treatment pathways can include the secondary treatment pathways. In some embodiments, the subset of the treatment pathways can include the treatment pathway containing the module to which the patient was most recently assigned. As may be appreciated, the linkages between treatment pathways can be represented as a tree, with the primary pathway being the root of the tree and the secondary pathways most distant from the primary pathway being the leaves of the tree. For example, when the patient is associated with treatment pathways 301, 303, 305, and 307, the root of the tree can be treatment pathway 301 and the leaves can be treatment pathways 305 and 307. In some embodiments, the subset of the treatment pathways can include the leaves of the tree (or optionally the leaves and root of the tree). To continue the prior example, the subset can include treatment pathways 305 and 307 (or optionally treatment pathways 301, 305 and 307).

In some embodiments, interactive opportunities can be selected using weights associated with the interactive opportunities. In some embodiments, for each module considered by the patient support platform, the patient support platform can determine an opportunity set. The patient support platform can select an available opportunity. In some embodiments, the patient support platform can select the available opportunity randomly. In other embodiments, the patient support platform can select the available opportunity with the largest (or smallest) corresponding weight. The patient support platform can then determine a sum of a weight for the available opportunity and the weights of opportunities already in the opportunity set. The patient support platform can then compare the sum to a threshold for the module. If the sum is less than the threshold, the patient support platform can add the selected opportunity to the opportunity set. The patient support platform can then select another opportunity and repeat the process. When there are no more available opportunities (or when the weight for the smallest weighted opportunity is still too large), the patient support platform can consider another module (and generate another opportunity set for that other module). Once the patient support platform has considered all the modules, the patient support platform can provide the opportunities in the opportunity sets for the modules (e.g., the union of the opportunities in the opportunity sets) to the patient system for display.

In some embodiments, the weights can be dynamic (e.g., time-varying or updated in response to patient actions). In some embodiments, the patient support platform can adjust the weights based on the duration a patient has been assigned to a module or the number of interactive opportunities that the patient has selected from the module. For example, the patient support platform can decrease the weights for interactive opportunities for a module when the patient has been assigned the module for a long time and yet not selected many interactive opportunities from the module (thus increasing the number of missions from that module provided to the patient). In some embodiments, patient support platform can adjust the weights for individual interactive opportunities (e.g., increasing the weight of an opportunity when similar opportunities have been selected previously), types of interactive opportunities, modules, or treatment paths.

In some embodiments, the patient support platform can determine a relative allocation among modules or among treatment paths through the relative threshold values among the modules or treatment paths. With reference to FIG. 1, for example, a threshold of 100 can be assigned to treatment path 301 and a threshold of 150 can be assigned to treatment path 303. Assuming individual missions in this example all have a weight of 10, the platform can provide to the patient 10 missions from the currently assigned module on treatment path 301 and 15 missions from the currently assigned module on treatment path 303. In some embodiments, the patient support platform can have an overall threshold that is broken out into threshold values for each module or treatment path associated with the patient. In some embodiments, the overall threshold can represent an assessment of the number of options that the patient should receive, to avoid decreasing engagement. The overall threshold can be constant; or can vary between patients or over time. With reference to FIG. 1, for example, the overall threshold of 100 can be split 60/40 between treatment path 301 and treatment path 303. Assuming individual missions in this example all have a weight of 10, the platform can provide to the patient 6 missions from the currently assigned module on treatment path 301 and 4 missions from the currently assigned module on treatment path 303.

In some embodiments, the patient support platform can select interactive opportunities using a predictive model. The predictive model can be a machine learning model (e.g., a neural network model, a reinforcement learning model, a decision tree or forest, or the like). The inputs to the predictive model can be or include portions of a profile for the patient (e.g., as described with regards to patient profiles 220, herein). In some embodiments, inputs to the model can include past patient interactions with the patient support platform. Such past patient interactions can include the record of past patient interactive opportunities, or the trajectory of the patient through one or more treatment pathways. For example, the predictive model can be a multi-armed bandit reinforcement-learning model that increments a likelihood of providing a type of interactive opportunity when the patient selects that interactive opportunity.

In some embodiments, during onboarding, configuration parameters (e.g., weights for missions, thresholds for treatment pathways, the distribution of an overall threshold between treatment pathways, or the like; or the parameters of a predictive model based on past patient interactions) for selecting missions can be determined based on patient medical information, demographic information, location, or the like. To determine the configuration parameters, the onboarding model can use data for a new patient and for the existing patients. For example, the onboarding model can be a nearest neighbor algorithm, clustering algorithm, neural network, decision tree or random forest, or similar statistical or machine learning model (or combination thereof).

Consistent with disclosed embodiments, a patient associated with multiple treatment pathways can progress along only one of the multiple treatment pathways at a time or can progress along two or more of the multiple treatment pathways simultaneously. In some embodiments, the patient may continue to progress along a pathway after being transferred to another pathway. For example, progression along the original pathway may be determined by elapsed time. As an additional example, progression along the original pathway may be determined by patient competencies, and other pathway may assist the patient in developing these competencies. As a further example, when the patient support platform selects among missions associated with multiple pathways, the user may continue to perform interactive opportunities associated with both pathways, thus causing the user to continue to progress along both pathways.

As part of an onboarding process, the patient support platform can associate a patient with a pathway and assign the patient to a module within that pathway. The patient support platform can, as part of this onboarding process, create or support creation of an account for the patient. Account creation can include entering patient information, indicating a treatment pathway (e.g., the rheumatoid arthritis pathway), providing information necessary for the platform to retrieve patient medical records, associating wearable devices (e.g., fitness trackers, smartwatches, or the like) with the account of the patient, or the like. In some embodiments, the account may be created at least in part by the patient, or by a person associated with the platform (e.g., an administrator or programmer of the platform, or the like). For example, the patient or the person associated with the platform can interact with the platform to create the account.

In hypothetical example depicted in FIG. 3A, the patient support platform assigns a patient diagnosed with Rheumatoid Arthritis to module 1 of the pathway 301. The patient support platform can perform this assignment as part of an onboarding process. Pathway 301 is configured to support patients with Rheumatoid Arthritis by providing them interactive opportunities for managing their condition. For example, module 1 can include educational missions describing the causes and symptoms of Rheumatoid Arthritis and medical and behavioral treatments for Rheumatoid Arthritis; physical missions intended to make the patient more active, and mental missions for combatting stress and concern the patient may have regarding their condition. Pathway 301 can be designed such that completion of the pathway would indicate that the patient is effectively managing their condition. In order to progress along pathway 301, a patient must satisfy a progression criterion for each module. For example, in order to progress to module 2, the patient may need to demonstrate a certain level of physical activity. In this example, pathway 301 can include a link 313a from module 1 to a pathway 303 for treating depression and a link 313c from module 3 to pathway 307 for encouraging medication adherence. A transfer condition, as described herein, can be associated with each of these links. Pathway 301 can also include a link 315 from module 3 back to module 2, to enable a patient to further develop competencies before progressing to module 4. A reassignment condition, as described herein, can be associated with link 315.

In this hypothetical example, module 1 of treatment pathway 301 includes a questionnaire configured to identify factors that might prevent a patient from achieving a suitable level of physical activity. Such a questionnaire could include questions concerning express inhibition on the patient's level of physical activity (e.g., whether the patient's mood prevents them from performing physical activities, or the like) or concerning potential inhibitions on the patient's level of physical activity (e.g., a depression diagnostic). In this example, the patient completes the questionnaire, and the resulting indicium of depression satisfies the transfer condition associated with link 313a. In response to completion of the questionnaire, upon completion of module 1, or the like, the patient support platform transfers the patient to module X of pathway 303.

Pathway 303 is configured to support patients with depression by providing them interactive opportunities for managing depression. In this example, pathway 303 is independent of pathway 301. For example, some patients may arrive at pathway 303 from other pathways and others may be initially associated with pathway 303 based on a diagnosis of depression. Pathway 303 can be designed such that completion of the pathway can indicate that a patient is effectively managing their depression. Like pathway 301, pathway 303 can include links to other pathways. In this example, pathway 303 include links to pathways concerning conditions contributing to depression (which may require management for effective treatment of depression). As depicted in FIG. 3A, module Y of pathway 303 includes a link 313d to pathway 305. Pathway 305 is configured to teach patients pain management techniques: completion of pathway 305 can indicate that a patient is effectively managing their pain. In response to competition of a mission (e.g., submission of a patient reported outcome indicating that pain is a cause of the patient's depression), the patient support platform transfers the patient to module I of pathway 305. The patient can then proceed along pathway 305.

In this hypothetical example, the patient support platform is configured to provide interactive opportunities from modules included in a subset of the treatment pathways associated with the patient. The subset includes the root and leaves of the tree formed by the links between the primary and secondary treatment pathways, and among the secondary treatment pathways. Thus, when the patient is assigned to module 1 of treatment pathway 301 and module Y of treatment pathway 303, the patient will receive interactive opportunities associated with module 1 and module Y. When the patient is assigned to module 3 of treatment pathway 301, module Y of treatment pathway 303, module II of pathway 305, and module A of pathway 307, the patient will receive interactive opportunities associated with module 3, module II, and module A (e.g., the root and leaves of the tree).

In this hypothetical example, the patient support platform is configured with an overall threshold of 100. The threshold is divided 40/30/30 between treatment pathways 301, 305, and 307. Module 3 includes two available interactive opportunities, a first mission with a weight of 41 and a second mission with a weight of 10. Module II and module A each include multiple interactive opportunities, each having a weight of 15. The patient support platform therefore selects the second mission from module 3, and two missions each from modules II and A to display to the patient. If the threshold were divided 80/10/10 between treatment pathways 301, 305, and 307, the patient support platform would select the first and second missions from module 3, and no missions from modules II and A to display to the patient.

While the treatment pathways depicted in FIG. 3A do not include branches, the disclosed embodiments are not so limited. For example, a patient assigned to module 1 of treatment pathway 301 could be given the option of progressing to module 2A or module 2B, each of which could have differing mixtures of associated interactive opportunities. For example, module 2A could offer a mix of educational and physical missions, while mission 2B could include only educational missions. Furthermore, module 2A could be linked directly to module 3. In contrast, module 2B could be linked to module 2C and module 2C could be linked to module 3. In this manner, patient choices could affect their progression through treatment, while remaining on the treatment pathway. This degree of patient choice can increase patient engagement with the platform.

FIG. 3B depicts an exemplary patient progression architecture based on flexible selection of missions within modules, in accordance with disclosed embodiments. The within-module flexibility depicted in FIG. 3B can be provided as an addition, or as an alternative, to the between-pathway flexibility described with regards to FIG. 3A, above. For convenience of explanation, only missions are depicted among the interactive opportunities for pathway 301 in FIG. 3B. But other interactive opportunities, such as questionnaires, data submissions, patient communications, or the like, could also be included. Furthermore, the number of interactive opportunities would typically be far greater than the number shown (e.g., the interactive opportunities could include 5 to 50 missions of various types). In this example, movement missions can include missions that require physical activity by the patient (e.g., walking a distance, doing a number of jumping jacks, etc.), mind missions can include missions that improve mental well-being (e.g., mindfulness exercises, mediation, or the like), and food missions can include missions that relate to proper eating choices (e.g., education about proper food choices, creating a food journal, uploading documentation of a meal to the platform, or the like).

Each module in treatment path 301 can be associated with interactive opportunities. In some embodiments, the associated interactive opportunities can include one or more mandatory interactive opportunities. In some instances, satisfaction of a progression criterion for the module can require completion of the mandatory interactive opportunities associated with that module. In the example depicted in FIG. 3B, module 3 is associated with mandatory mission 325a. Completion of mandatory mission 325a may be necessary for progression from module 3 to module 4. In some instances, completion of such mandatory interactive opportunities can be sufficient for progression, while in other instances addition conditions may need to be satisfied. For example, a mandatory interactive opportunity may be a test designed to evaluate a patient's knowledge of their condition. Progression to the next module may require completion of the test and a sufficiently high score on the test. As an additional example, progression to the next module may require completion of any mandatory interactive opportunities, and completion of a set number of other interactive opportunities. In some instances, progression may not require completion of specific mandatory missions. Instead, progression can require completion of a number of missions, expiration of a time period (e.g., each week the patient can progress to the next module), or satisfaction of another suitable condition.

In some embodiments, the patient support platform can select among available interactive opportunities for interactive opportunities to provide to the patient. In some instances, an available interactive opportunity can become unavailable once completed by the patient, preventing it from being selected again. In various instances, an available interactive opportunity can remain available after being completed by the patient, allowing the patient the opportunity to complete it again. In some embodiments, the patient support platform always selects available mandatory opportunities for display. The patient support platform may also select additional opportunities as described herein (e.g., using weights and thresholds, a predictive model, or the like).

In some embodiments, dependencies can impose a performance order on two or more interactive opportunities associated with a module. An interactive opportunity may not be available for provision to the patient until the patient completes the interactive opportunit(ies) upon which it depends. In the example depicted in FIG. 3B, food mission 325d may not be available for provision to the patient until the patient completes food mission 325c. Likewise, food mission 325c may not be available for provision to the patient until the patient completes movement mission 325b and mandatory mission 325a.

In some embodiments (not depicted in FIG. 3B) an interactive opportunity within a module can be gated by a competency requirement. For example, the interactive opportunity may not be available until the competencies of the patient satisfy a condition (e.g., a level of a competency exceeds a threshold, or the like).

FIG. 3C depicts an exemplary method 300 of mastery-based progression through the patient architectures depicted in FIGS. 3A and 3B, in accordance with disclosed embodiments. Method 300 can include a feedback loop in which the patient support platform provides, based on competencies of the patient, an interactive opportunity to a patient. Method can be performed by an application system (e.g., application system 101) operating in conduction with data storage (e.g., data storage 103) to communicate with a patient system (e.g., one of patient system(s) 105) and receive information from patient wearable device(s) (e.g., a subset of patient wearable device(s) 107). The disclosed embodiments are not limited to any particular format or network for such communications. In response to completion of the interactive opportunity by the patient, the patient support platform updates the competencies of the patient. In this manner, the patient support platform can continue to provide interactive opportunities appropriate for individual patients, even over time or as those patients progress through their treatment. By providing interactive opportunities of appropriate difficulty to the patient, the patient support platform can increase patient engagement.

In step 331 of method 330, the patient support platform can obtain an interactive opportunity, consistent with disclosed embodiments. The patient support platform can select the interactive opportunity from among available interactive opportunities associated with a module currently assigned to the patient. Consistent with disclosed embodiments, the interactive opportunity can be mandatory or optional. Additionally, the module can be included in a treatment pathway currently associated with the patient.

In step 333 of method 330, the patient support platform can provide the interactive opportunity to the patient, consistent with disclosed embodiments. The disclosed embodiments are not limited to any particular format, protocol, or network for providing the interactive opportunity. For example, the data and instructions could be provided using HTML, JSON, Javascript, or any other suitable method. The data or instructions can be adapted to the patient system. The data or instructions can enable the patient system to display the interface for performing (and optionally for configuring) the interactive opportunity.

In step 335, the patient support platform can obtain performance data for the interactive opportunity, consistent with disclosed embodiments. For example, such performance data can be pushed to the patient support platform or retrieved from a source external to the patient support platform. The specific performance data obtained can depend on the interactive opportunity and therefore the disclosed embodiments are not limited to receiving performance data of a particular type or format, using a particular network. In various embodiments, the performance data can include a indication that an interactive opportunity was completed (or not completed) by the patient; data received from the patient system (e.g., data entered into fields on a user interface of the patient system, form data including answers to a questionnaire, or the like); data (e.g., activity or biometric data) obtained by a wearable device of the patient (e.g., one of patient wearable device(s) 107), such as heartrate, number of steps, distance traveled, EEG values, pulse oximetry, breathing rate; or the like.

In some embodiments, patient support platform can communicate with the patient system regarding the interactive opportunity. Such communication can occur before, during, or after performance of the interactive opportunity. As part of such communication, the patient support platform can receive indications that the patient is performing the opportunity (e.g., selection of a “start” control, or the like), configuration instructions for the opportunity (e.g., selection of “weight” and “age” values for a physical mission intended to safely elevate the heart-rate of the patient, or the like), requests for content involved in performance of the opportunity (e.g., video content for an education opportunity, questions for a questionnaire, or the like), or other suitable indications. The patient support platform can provide additional data or instructions in response to such indications (e.g., data or instructions for updating the patient system user interface to acknowledge start of an opportunity, to display a safe heart rate based on the age and weight of the patient, or to display or stream requested content.

In step 337, the patient support platform can update competency information for the patient (e.g., competency information 225) based on the obtained performance data. The performance data can implicate multiple competencies tracked by the patient support system for the patient. For example, completion of a goal-setting interactive exercise could implicate a treatment-engagement competency (as it involves patient interactions with the platform), as well as other competencies that concern the goals set (e.g., setting a goal to abstain from meat for a week could implicate a diet management competency). In some embodiments, the interactive opportunity can specify the competencies implicated by the performance data. Additionally, or alternatively, the interactive opportunity can specify relationship(s) between the performance data and the implicated patient competenc(ies). For example, performance data concerning a running mission can include average pace for the mission. The interactive opportunity can specify a binning function or the like that maps this average pace to a new physical competency level of the patient or an update to an existing competency level of the patient.

As may be appreciated, a patient may be performing multiple interactive opportunities simultaneously (e.g., walking while listening to a podcast on treatments for their condition). Accordingly, in some instances, the patient support platform can be configured to update competency information once an indication has been received that performance of an interactive opportunity is complete.

In step 339, the patient support platform can update the progression of the patient. In some embodiments, such updating can include determining whether a progression criterion or reassignment criterion for reassigning the patient to a new module is satisfied or determining whether a transfer criterion for transferring the patient to another treatment pathway is satisfied. In various embodiments, such updating can affect whether new interactive opportunities within the present module are available. For example, the module can include data or instructions specifying that a physical mission associated with the module be unavailable unless the patient has a level of physical competency greater than a threshold value. The update performed in step 337 can cause the patient's level of physical competency to exceed the threshold. Because the physical mission is now available, the patient support platform can now select the physical mission for provision to the patient. In some embodiments, data or instructions associated with the physical mission can be updated to indicate that the mission is now available. In various embodiments, determining whether a mission is available can be performed when selecting missions (e.g., step 331, or the like).

In some embodiments, in step 341, the patient support platform can provide progression options to the patient system. For example, the treatment pathway may permit progression from the current module to one of two subsequent modules. This progression can be governed by a progression criterion or criteria. The patient support platform can determine in step 339 that the progression criterion or criteria were satisfied. The patient support platform can then enable the patient to chose which of the two modules should be assigned to the patient. For example, the patient support platform can provide data or instructions to the patient system to display the choice. Similarly, the patient may have the option of progressing along the current treatment pathway or transferring to another treatment pathway or being reassigned to a prior module or remaining at the current module.

In some embodiments, in step 343, the patient support platform can receive a selection of one or more progression options from the patient. The patient support platform can update the progression of the patient in the patient support platform based on the selected progression option(s). For example, the patient support platform can assign the patient to a selected new module or a new treatment pathway or return the patient to an earlier module.

After the patient support platform has completed updating the progression of the patient (and potentially resolving progression options), the patient support platform may return to step 331 and obtain a new interactive opportunity based on the updated progression of the patient in the patient support platform.

FIG. 4 depicts a view 400 of an exemplary user interface for providing personalized treatment support, in accordance with disclosed embodiments. View 400 can be displayed by a patient system (e.g., one of patient systems 105), based on data or instructions received from an application system (e.g., application system 101). View 400 is relatively simple and small, two factors that can decrease engagement. However, user interface 400 can be configured, or the content displayed in user interface 400 can be selected, to address these technical problems.

Display 401 can be a control configured to display a selection of interactive opportunities received from the patient support platform. These interactive opportunities can be selected as described herein with regards to FIG. 3A, FIG. 3B, and FIG. 3C. The selection process can increase the likelihood that the displayed interactive opportunities are relevant to the patient and suitable for their current competency levels, thus improving engagement. As displayed in FIG. 4, display 401 can be a scroll box having selectable elements corresponding to each interactive opportunity. The patient can select an element to transition to another view to customize or initiate performance of that interactive opportunity. The disclosed embodiments are not limited to such a scroll box: other graphical elements or controls can be used to display the selected interactive opportunities without departing from the envisioned embodiments.

View 400 can also include gamification elements to drive user engagement. Such gamification elements can include progress indicators 403a, 403b, and 403c. Such indicators can provide immediate feedback on what needs to be done to complete a task or earn a reward. For example, in some embodiments patient actions can result in clean water (or tablets for cleaning water) being donated to people in developing countries. The patient support platform can track the amount of water donated as a result of the patient's actions. Progress indicator 403a can fill up as this amount increases, emptying when the patient increases in level. This provides an immediate indication of an altruistic reward, to motivate the patient. Progress indicator 403b similarly indicates the progress of the patient in fulfilling a progression condition for a module (e.g., based on the number of interactive opportunities completed, or the like). In this example, the patient support platform also indicates the progress of the patient in fulfilling an interactive opportunity (in this instance a physical mission for walking a certain number of steps), using progress indicator 403c. Collectively, these indicators demonstrate the patient's achievements and goals at different levels, from immediate (e.g., progress indicator 403c) to long-term (e.g., progress indicator 403a).

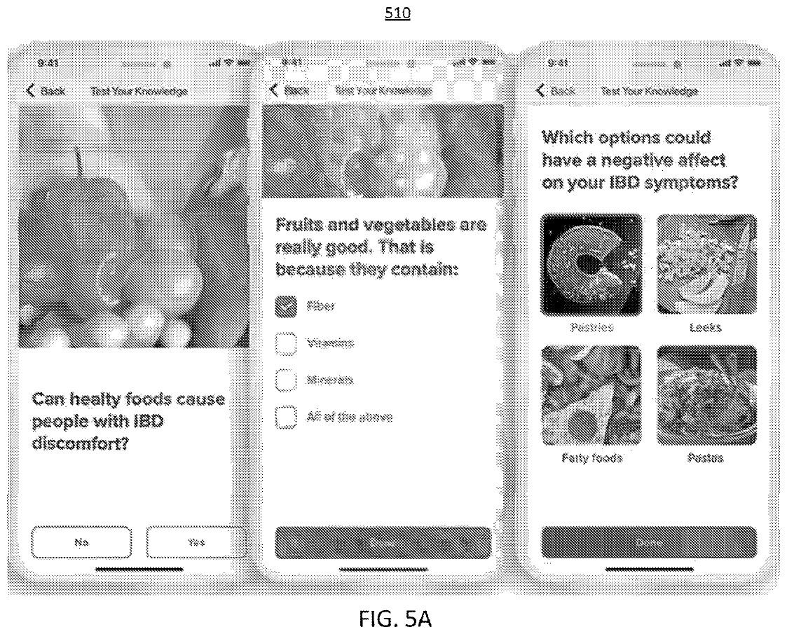

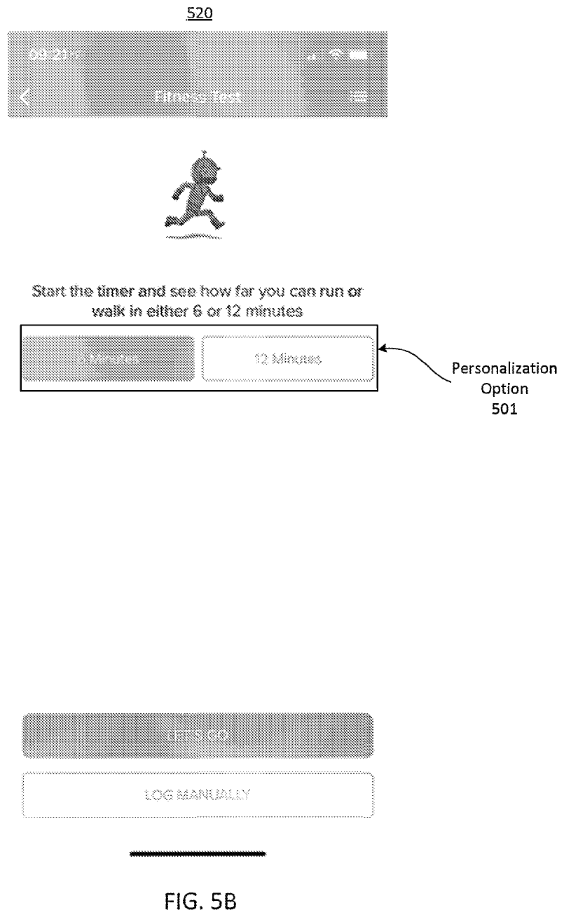

FIG. 5A depicts interactive opportunity completion views 510 of an exemplary user interface for providing personalized treatment support, in accordance with disclosed embodiments. In some embodiments, a patient can reach such views for respective interactive opportunities by selecting elements of display 401 in view 400 that correspond to the interactive opportunities. Views 510 display exemplary interactive opportunities. The patient can interact with each view to complete the corresponding opportunity. In these examples, the interactive opportunities constitute short quizzes that gauge the patient's understanding of their condition. Good performance on such interactive opportunities may be necessary for satisfying a progression criterion and being assigned to the next module in a treatment pathway. Poor performance on such interactive opportunities may satisfy a reassignment criterion, causing the patient support platform to reassign the patient to an earlier module in the pathway (and thus causing the patient to repeat educational missions concerning their condition). As can be appreciated, such changes in progression would affect the interactive opportunities subsequently provided to the patient.

FIG. 5B depicts an interactive opportunity customization view 520 of an exemplary user interface for providing personalized treatment support, in accordance with disclosed embodiments. In some embodiments, a patient can reach such a view for an interactive opportunity by selecting an element of display 401 in view 400 that corresponds to the interactive opportunity. View 520 enables the patient to select personalization options (e.g., personalization option 501), thereby customizing this interactive opportunity. In this instance, the interactive opportunity is a physical mission. Completing the physical mission requires walking for a specified duration. The personalization option controls specify this duration. As described herein, the patient support platform can track patient interactions. In some embodiments, the tracked interactions can be used to set default values for such personalization option controls. In this example, the option value selected now can affect the option values presented the next time this patient selects this mission (or a similar mission). For example, if the patient selects 12 minutes now and successfully completes the mission, then the option values presented next time can be 12 and 18 minutes. Or the 12-minute option can be the default, rather than the 6-minute option. In some embodiments, when a patient is being onboarded, default values for such personalization option controls can be predicted using a nearest neighbor algorithm, clustering algorithm, neural network, decision tree or random forest, or similar statistical or machine learning model (or combination thereof).

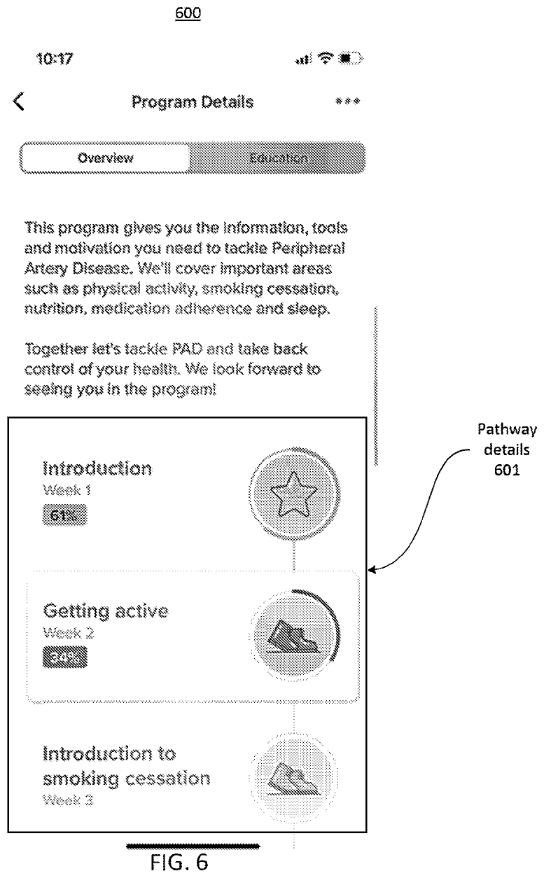

FIG. 6 depicts a pathway information view 600 of an exemplary user interface for providing personalized treatment support, in accordance with disclosed embodiments. In some embodiments, a patient can reach such a view by selecting a suitable control in view 400. View 600 can display information about the modules in a pathway. In this example, pathway details 601 includes selectable elements corresponding to the modules in the pathway. The selectable elements can include gamification features, such as progression indicators, to further patient engagement. There are three modules in this example and the progression criterion for each module is time-based (e.g., a week for each module).

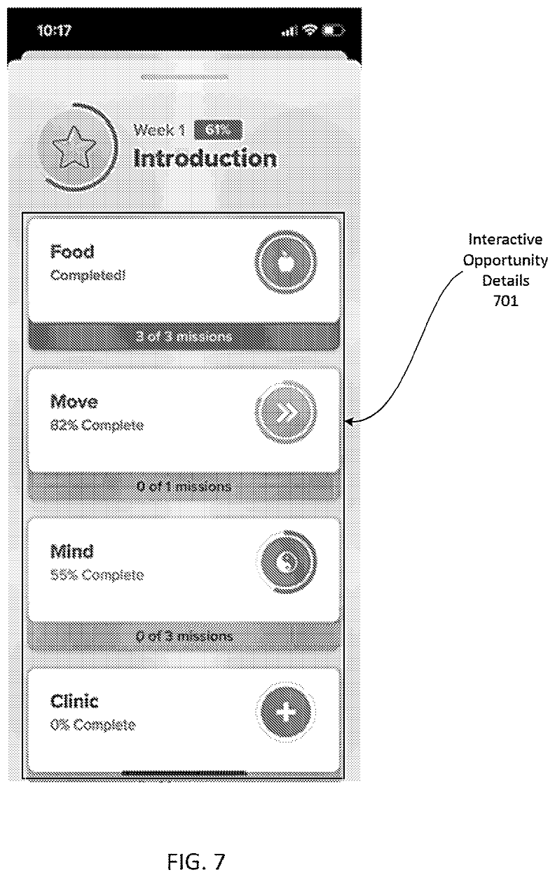

FIG. 7 depicts a module information view 700 of an exemplary user interface for providing personalized treatment support, in accordance with disclosed embodiments. In some embodiments, a patient can reach such a view for a module in a pathway by selecting an element of pathway details 601 in view 600 that corresponds to the module. View 700 can display information about the categories of interactive opportunities available, and the numbers of interactive opportunities of each category. In this example, interactive opportunity details 701 can be a scroll box having selectable elements corresponding to the categories of interactive opportunities in the module. The selectable elements can include gamification features, such as progression indicators, to further patient engagement. There are four categories of interactive opportunities in this example: food (e.g., dietary, nutrition, or the like) missions, move (e.g., physical) missions, mind (e.g., meditation, mindfulness, mental health, or the like) missions, and clinic missions. These categories are not intended to be limiting.

In some embodiments, the exemplary user interface for providing personalized treatment support can include an interactive opportunity category information view. In some embodiments, a patient can reach such a view for an interactive opportunity category in a module in a pathway by selecting an element of interactive opportunity details 701 in view 700 that corresponds to the interactive opportunity category. The view can display information about each of the interactive opportunities in the category for the module. This information can include whether each interactive opportunity has been completed, is being completed, is available for completion, is unavailable for completion, any requirements (e.g., competency requirements) for selecting the interactive opportunity, any rewards or competency effects of completing the mission, or the like.

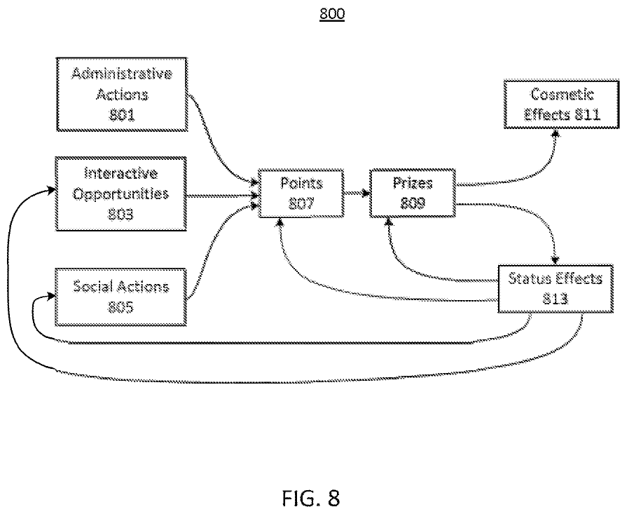

FIG. 8 depicts an exemplary game loop 800 for an application for providing personalized treatment support, in accordance with disclosed embodiments. The game loop can increase the patient's engagement by, for example, rewarding desired patient actions, building a sense of achievement or investment, or satisfying the patient's desire for social recognition.

Consistent with disclosed embodiments, game loop 800 can include performance of actions by the patient. In some embodiments, the actions can include maintenance actions 801, interactive opportunities 803, and social actions 805. Administrative actions 801 can include patient actions directed to the patient support platform, such as logging in, creating an account, linking a patient management system or wearable device for the patient to the platform, or other administrative interactions with the platform that do not concern the competition of interactive opportunities. As described herein, interactive opportunities can include missions, questionnaires, data submissions, patient communications, or the like. In some embodiments, the patient support platform can include social media elements, such as public pages, timelines, posts, or the like. Patients using the patient support platform can communicate with each other and the overall patient community using such social media elements. Social actions 805 can include adding content using the social media elements (e.g., the patient adding a post, updating a page of the patient, commenting on or replying to content added by another patient, or the like), sharing content using the social media elements (e.g., forwarding content inside or outside the patient support platform), reviewing or “liking” content in the patient support platform, or the like.

In some embodiments, the patient support platform can provide forums, rankings, and other community-building social elements. For example, the patient support platform can provide leaderboards, or support forums devoted to particular conditions or treatments. Patients can obtain points 807 for placing on a leaderboard or participating in a forum.

Consistent with disclosed embodiments, the patient support platform can provide points 807 to the patient for completing actions. In some embodiments, points 807 can take the form of one or more in-platform currencies. In some embodiments, different types of actions can be associated with different types of in-game point types. For example, each of maintenance actions 801, interactive opportunities 803, and social actions 805 can be associated with a different in-game point type. In some embodiments, different types of interactive opportunities can provide different point types (or differing combinations of point types).

Consistent with disclosed embodiments, the patient can obtain prizes 809 using points 807. Prizes 809 can be varied in type, cost, potential contents, or other suitable characteristics. In some embodiments, a prize can be a virtual container that, when opened, reveals a benefit to the patient (e.g., a “loot box” or the like). Such benefits can include cosmetic effects 811 and status effects 813.

Cosmetic effects 811 can include badges (e.g., representation of achievement that can be added to the patient's user interface). In some embodiments, the patient support platform can update the user interface through which the patient interacts with the platform, or update a social media page, profile, or timeline associated with the patient, to display badges earned by the patient. For example, the platform can display a virtual laboratory, a virtual tree, and a virtual fountain. A prize can include a “vaccine drop.” Obtaining enough such drops can cause the virtual laboratory to “level-up”, assuming an enhanced visual appearance. Likewise, a prize can include a “seed.” Obtaining enough such seeds can cause the virtual forest to “level-up”, assuming an enhanced visual appearance. Likewise, a prize can include a “water drop” or “purification tablet.” Obtaining enough such drops or tablets can cause the virtual fountain to “level-up”, assuming an enhanced visual appearance.

In some embodiments, real world actions can be associated with prizes. For example, one or more entities (e.g., the entity controlling the patient support platform) can agreed to perform real world actions in response to the patient obtaining prizes. For example, the one or more entities can commit to donating a number of vaccine doses based on the number of “vaccine drops” obtained by the patient, planting a number of trees based on the number of “seeds” obtained by the patient, or donating a quantity of water based on the number of “tablets” or “water drops” obtained by the patient. Thus, the patient can achieve altruistic goals through interactions with the platform. In this manner, the badges (e.g., the virtual laboratory, virtual tree, or virtual fountain) can become lasting representations of the patient's achievement of these altruistic goals.

Status effects 813 can affect the operation of the patient support system or game loop 800. Such effects can be limited (e.g., in time or to a certain number of uses) or permanent. Status effects 813 can affect the social actions 805 that can be performed by the patient using social media elements of the platform. For example, a status effect could allow the patient to act as a moderator of a forum concerning their condition, or coach other patients having the same condition (and could unlock additional functionality enabling them to do so). Status effects 813 can affect the interactive opportunities 803 available to the patient. For example, status effects could temporarily allow a patient to attempt interactive opportunities that would otherwise require a higher level of competency, redo interactive opportunities and attempt to achieve higher scores, attempt otherwise unavailable “locked” or “hidden” interactive opportunities that provide special rewards, or the like. Status effects 813 can affect points 807 obtained for completing actions by increasing the number of points obtained. For example, a status effect could temporarily double the number of points obtained for completing interactive opportunities of a certain category. Status effects 813 can affect points prizes 813 by increasing the likelihood that a given prize contains benefits of a certain type or rarity, or by unlocking a new type or level of cosmetic effect. As can be understood from the forgoing, many types and combinations of status effects are possible. The disclosed embodiments are not limited to those particular examples enumerated above. Furthermore, cosmetic effects 811 and status effects 813 have been described separately for convenience, as the disclosed embodiments do not necessarily impose a rigid distinction between these general categories of benefits. For example, changes to the operation of the patient support system or game loop 800 (e.g., a status effect) can be signified or represented by a badge (e.g., a cosmetic effect).

In some embodiments, prizes 809 may be omitted. Instead, points 807 can be used directly to obtain desired benefits (e.g., cosmetic effects 811 and/or status effects 813). Similarly, in some embodiments, points 807 may be omitted. Instead, the patient may be awarded prizes 809 directly in response to completing actions.

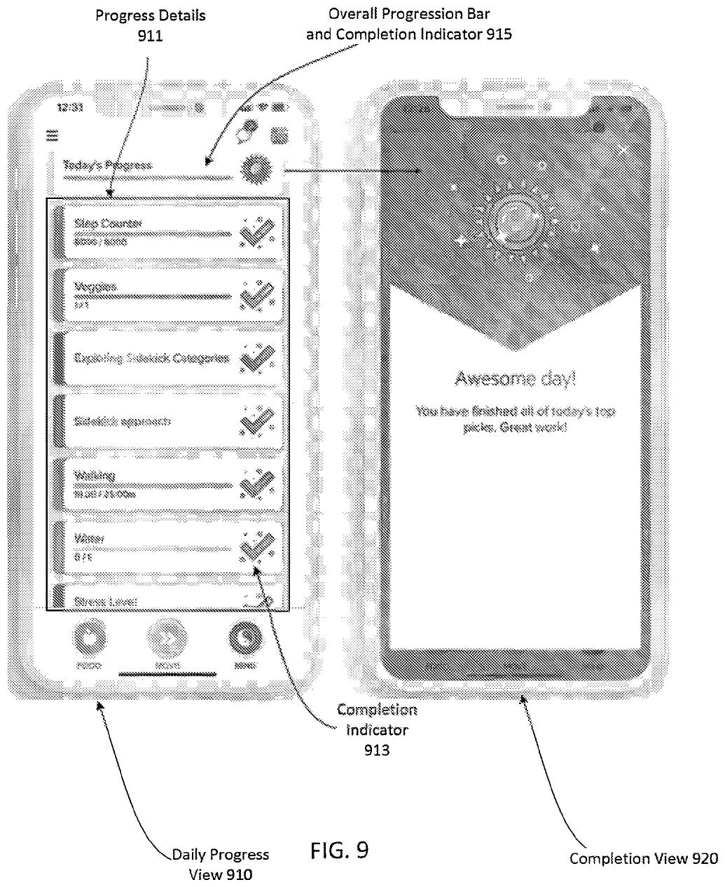

FIG. 9 depicts a daily progress view 910 and a completion view 920 of an exemplary user interface for providing personalized treatment support, in accordance with disclosed embodiments. In some embodiments, a patient can reach such a view for a module in a pathway by selecting a control in view 400. View 910 can display the progress achieved by a patient in the current day. In this example, progress details 911 includes selectable elements corresponding to interactive opportunities completed in the current day. In some embodiments, each element can include information about the completed interactive opportunity (e.g., the number of steps taken/required or the duration walked/goal duration in a physical mission). In some embodiments, each element can include a completion indicator (e.g., completion indicator 913) to affirm to the patient that the interactive opportunity has been completed.

In some embodiments, progress view 910 can include an overall progress bar and completion indicator 915 (in this example a clouded sun that progressively becomes unclouded as the patient completes interactive opportunities). The progress bar and indicator can show the patient how much overall work remains to complete a daily goal.

In some embodiments, upon completion of the interactive opportunities for the day, view 910 can transition to completion view 920. Completion view 920 can provide encouraging text or visuals to reward the patient for completing the allotted interactive opportunities.

FIG. 10 depicts views associated with opening a prize, in accordance with disclosed embodiments. View 1010 depicts a completion screen for an interactive opportunity. In some embodiments, a patient can reach this view by competing the interactive opportunity. In this example, rather than being awarded points that can be used to obtain prizes, the patient is awarded the prize directly. View 1010 includes claim control 1011, which the patient can select to claim a prize. View 1020 is an interstitial view and serves to build anticipation in the user for the prize. In this example, the prize is an amount of clean water to be donated by the entity controlling the patient support platform. The interstitial view indicates the amount of clean water already donated (or to be donated) as a result of the patient's actions. View 1030 depicts the amount of the prize (in liters of clean water). In some embodiments, the amount of the prize can be symbolically added to the existing total through a short animation and the incrementing of the existing total (e.g., from 315 liters to 325 liters).

FIG. 11A depicts a view 1110 of a coach portal interface for providing personalized treatment support, in accordance with disclosed embodiments. View 1110 can enable a coach to interact with one or more patients, addressing the lack of personal contact that afflicts existing patient support platforms. In some embodiments, the coach portal can be web-based. Application system 101 can be configured to provide data or instructions to coach system(s) 109 to cause these system(s) display instances of the coaching portal. View 1110 can support improved scalability by enabling a coach to simultaneously (e.g., in a single view) review the statuses and actions of multiple patients.

View 1110 can, in the example depicted in FIG. 11A, include a scroll box containing elements corresponding to patients (e.g., patient element 1111). Each element can display the name of the patient, outstanding actions concerning the patient (e.g., action items 1113), and indicators showing measures of the patients' engagement with the platform (e.g., engagement measures 1115a, 1115b, and 1115c). In this manner, the coach is presented with information indicative of the patient's engagement and any outstanding actions that the coach may be able to take to increase this engagement (or otherwise address patient needs). In this example, engagement measure depicts the interactive opportunities completed by the patient. The length of the bar can indicate the number of interactive opportunities completed. The sub-bars of different lengths and colors making up engagement measure 1115a can indicate the number of different categories of interactive opportunities completed. Engagement measure 1115b can depict the result of the most recent PRO (and patient element 1111 can also display how recently the PRO was submitted). Engagement measure 1115c can depict indicators for actions that must be completed by the coach for the patient (from left to right: evaluating a meal diary entry, evaluating a PRO, and responding to a communication).

In some embodiments, view 1110 can include user statistics 1117, an indicator that displays the total number of patients assigned to the coach, as well as the percentages of those patients that are presently active (e.g., interacted with the patient support platform within a certain period of time, such as the last week), that are new (e.g., added to the platform within a certain period of time, such as the last week), or that are inactive (meeting neither of the preceding conditions).

In some embodiments, view 1110 can include filter control 1119. Filter control 1119 can enable a coach to select an individual patient, or a class of patients. For example, the coach can filter the patient elements displayed in view 1110 by name by entering a complete or partial patient name into a search box, by activity status by selecting one or more activity statuses from a drop-down menu (e.g., new, active, inactive, or the like), by completion date of most recent PRO, by value (e.g., on a 1 to 10 scale, or the like) or the most recent PRO, by whether there are outstanding actions to respond to by selecting patient activity status from a drop-down menu, or a combination of the foregoing patient characteristics or similar patient characteristics.

In some embodiments, view 1110 can include a bulk message control 1121 that enables the coach to send messages to multiple patients. In some embodiments, the coach can interact with filter control 1119 to display a set of patients, then select bulk message control 1121 to open bulk message window 1123 (depicted in FIG. 11B). Bulk message window 1123 can be a pop-up window, though the disclosed embodiments are not limited to such an implementation. Once the coach hits send in bulk message window 1123, the message will be sent to all of the patients in the set. The coach can add additional recipients by adding their names to the address bar in bulk message window 1123. In this example, the coach can also send messages to individual patients by selecting their names.

Accordingly, view 1110 can provide a centralized interface for reviewing patient engagement, addressing outstanding patient needs, and communicating with patients, individually or in groups.

FIG. 11C depicts a patient information view 1130, in accordance with disclosed embodiments. A coach can select a patient element in view 1110 to transition to a patient information view for the patient. Patient information view 1130 can display patient information for the patient (in this example, weight, blood pressure, heart rate, cholesterol, blood sugar, BMI, and a value of an index of work-related stress). The patient support platform can code or categorize items of patient information, determining whether the values of such items indicate a potential health problem. In some embodiments, the patient support platform can apply general clinical guidelines in making such determinations (e.g., categorizing a blood pressure value of 120 to 129 mmHg systolic and less than 80 mmHg diastolic as elevated). In various embodiments, the patient support platform can use information about the patient to provide patient-specific assessments. For example, a decrease in sleep may result in an increase in blood pressure. The patient support platform may track the sleep schedule of a patient and determine that, in addition to having elevated blood pressure, the patient needs to sleep more. Thus, the patient support platform may indicate both blood pressure and sleep schedule as areas of concern for this patient. In some embodiments, such a determination by the patient support platform can depend on clinical outcomes or actions taken by patients managed by the patient support platform that are similar to this patient (e.g., using a clustering algorithm, categorization model, or the like). Automated indicators (e.g., automated indicator 1134) can indicate the results of this determination, emphasizing items of patient information that may need to be addressed by the patient (in this example blood pressure, cholesterol, and work-related stress). Selecting one of information tabs 1131 can cause the user interface to display a PRO feedback view 1200, a performance view 1300, or a food journal view 1410. Information view 1130 can include an indicator (e.g., treatment pathway 1133) that displays information regarding treatment pathways and modules associated with the patient. In this example, treatment pathway 1113 indicates that this patient is on module 3 of 16 of a Crohn's disease treatment pathway.

In some embodiments, message control 1135 can be selected by the coach to view and respond to messages provided by the patient. In this example, the indicator on this control indicates that the patient has sent a message and the coach needs to respond. FIG. 11D depicts a message window 1140 for communications between a coach and a patient, in accordance with disclosed embodiments. The coach can open window 1140 by selecting message control 1135 in view 1130. The patient and the coach can communicate using message window 1140, as shown.

FIG. 12 depicts a PRO feedback view 1200 of a coach portal interface for providing feedback on patient reported outcomes, in accordance with disclosed embodiments. As may be appreciated, a role of the coach is to guide patients to an accurate assessment of their own condition. When a patient submits a PRO survey describing their condition, an indication of their overall reported condition appears in view 1110 (e.g., engagement measure 1115b). In some instances, a coach can select that indication to transition from view 1110 to view 1200. Alternatively, a coach can select the PRO tab of information tabs 1131 in patient information view 1130 to transition to view 1200. View 1200 can provide a tabbed display of PRO results. Tabs can correspond to surveys (e.g., IBD symptom survey, living with Crohn's survey, intake survey, or the like) and selection of a tab can cause view 1200 to display the PRO results for that survey. The patient can take surveys multiple times. In this example, each column corresponds to one submission of the survey. In some embodiments, the patient can interact with view 1200 to view earlier dates (e.g., by paging the displayed dates to the right, removing the rightmost column, and adding a new leftmost column with an earlier date) or later dates (e.g., by paging the displayed dates to the left, removing the leftmost column, and adding a new rightmost column with a later date). View 1200 can indicate that a submission requires review (in this example through placement of a dot on the margin of the result indicator). The color and number depicted indicate the severity of the patient's condition. The coach can select the indicator for the submission requiring review and either approve or change the overall rating (e.g., to better match the self-reported symptoms). The coach has the option to also send a message to the patient. If the coach changes the rating, the color and number change, but an outer ring of the original color can be retained to indicate the original rating and that the rating was changed by the coach. This feedback can be visible to the patient and can increase patient engagement by demonstrating that their actions are being observed and reviewed by a person who wants to help them. This feedback can also help the patient provide an accurate assessment of their own condition.

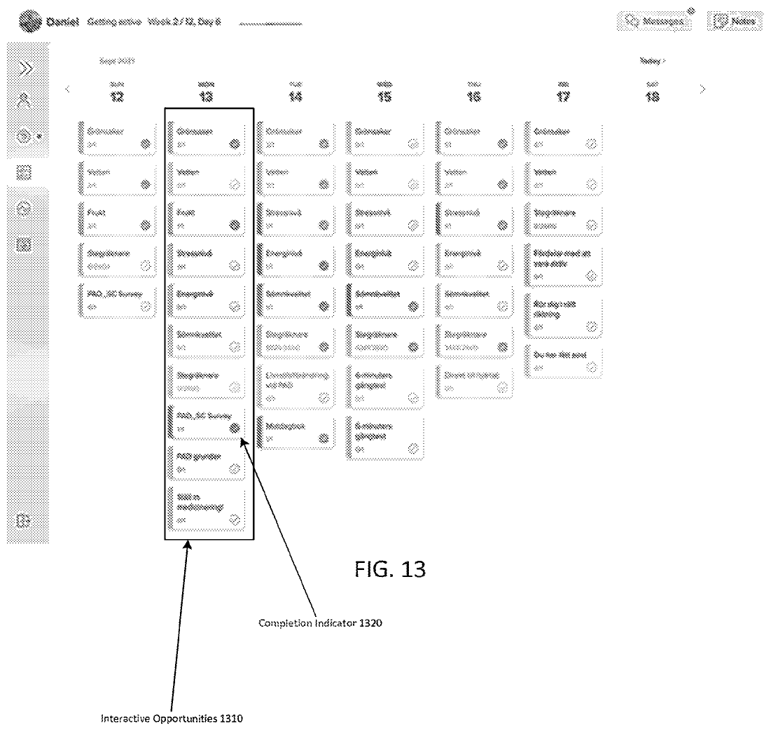

FIG. 13 depicts a performance view 1300 of a coach portal interface for reviewing user mission performance, in accordance with disclosed embodiments. A coach can select the performance tab of information tabs 1131 in patient information view 1130 to transition to view 1200. View 1300 depicts a day-by-day list of the interactive opportunities for the patient (e.g., interactive opportunities 1310). In some embodiments, these opportunities may have been selected by the patient from among those interactive opportunities provided by the patient support platform. In various embodiments, they may have been directly assigned by the patient support platform. Those interactive opportunities completed by the patient may be indicated in view 1300 (e.g., by a completion indicator such as completion indicator 1320). In some embodiments, the patient can interact with view 1300 to view earlier dates (e.g., by paging the displayed dates to the right, removing the rightmost column, and adding a new leftmost column with an earlier date) or later dates (e.g., by paging the displayed dates to the left, removing the leftmost column, and adding a new rightmost column with a later date). In this manner, view 1300 can present the coach with a comprehensible overview of what missions the patient is attempting and what missions the patient is completing. This information can enable the coach to address any weaknesses in the patient's performance (e.g., a failure to select many missions, or a failure to complete missions of certain types, or a progressive decrease in the number of missions performed over the course of the week).

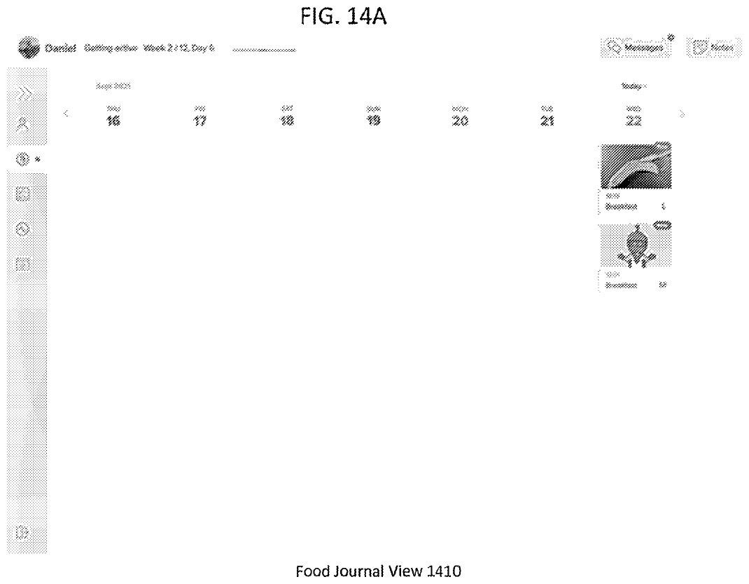

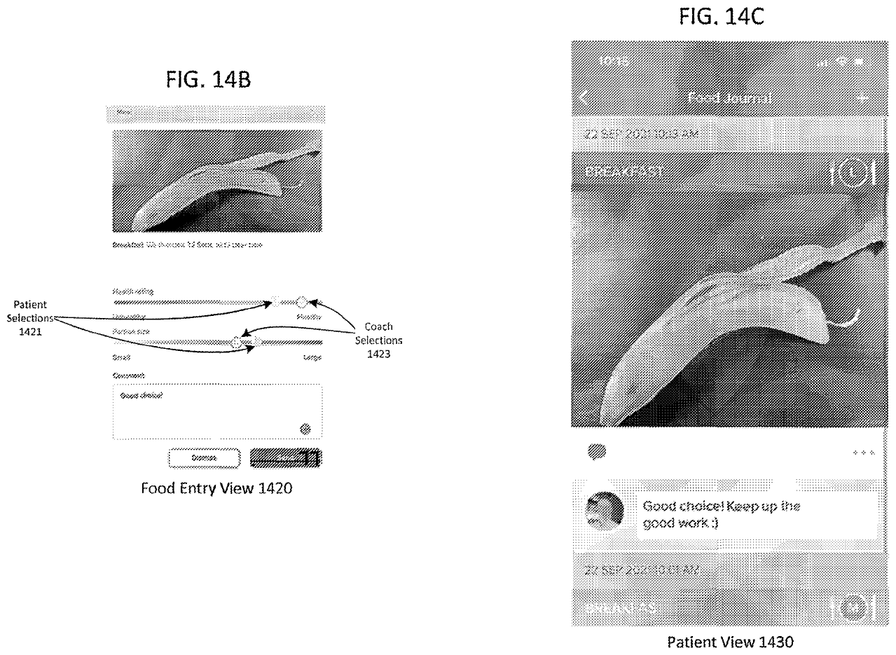

FIG. 14A depicts a food journal view 1410 of a coach portal user interface for reviewing user food journals and providing feedback, in accordance with disclosure embodiments. When a patient submits a food journal documenting a meal, an action appears in the patient element for that patient (e.g., action items 1113 of patient element 1111 includes as the first action item an indication that a food journal requires review). In some instances, a coach can select that indication to transition from view 1110 to view 1410. Alternatively, a coach can select the food journal tab of information tabs 1131 in patient information view 1130 to transition to view 1410. View 1410 can depict a day-by-day list of food journal entries submitted by the patient. Each column can be a day, with column entries for each food journal submission. View 1410 can indicate unreviewed entries (e.g., with the label “new” as shown). In some embodiments, the patient can interact with view 1410 to view earlier dates (e.g., by paging the displayed dates to the right, removing the rightmost column, and adding a new leftmost column with an earlier date) or later dates (e.g., by paging the displayed dates to the left, removing the leftmost column, and adding a new rightmost column with a later date). In this manner, view 1410 can present the coach with a comprehensible overview of what the patient is eating (or whether the patient need to be more conscientious in documenting their meals).

FIG. 14B depicts a food entry view 1420 of a coach portal user interface for reviewing an individual food journal submission, in accordance with disclosed embodiments. In some embodiments, the view includes an image taken by the patient of the meal of the patient. The patient can upload this image to the platform. The patient can also select values for two meal characteristics: health rating and portion size (e.g., patient selections 1421). In some embodiments, the coach can adjust these values (e.g., coach selections 1423). This feedback can increase patient engagement by demonstrating that their actions are being observed and reviewed by a person who wants to help them. In this example, coach selections 1423 show that the user overestimated the size and underestimated the healthiness of their breakfast. In some embodiments, the coach can also provide a comment to the user. This comment functionality can explain the adjustments made by the coach and provide support and encouragement to the patient.

FIG. 14C depicts a patient view 1430 of a patient user interface, in accordance with disclosed embodiments. Patient view 1430 can appear on a display of a patient system (e.g., one of patient systems 105). Patient view 1430 can appear in response to submission by a coach of a comment or review regarding a food journal entry of the patient. In this example, patient view 1430 displays the image from the food journal and the comment from the coach.

As may be appreciated from the foregoing, coaching patients may require substantial involvement from the coach. When a coach is responsible for many patients, responses to patient actions may be delayed. Such delays may risk causing a patient to disengage from the platform. To address this risk, the patient support platform can be configured to automatically review patient actions, or to automatically respond to patient communications. For example, the platform can determine that an unreviewed patient action or communication satisfies a delay criterion (e.g., a delay greater than a threshold value, or the like). The platform can generate a responsive message. In some embodiments, the message can be predetermined and general (e.g., “Thank you for logging your meal. Keep up the good work!”). In various embodiments, the message can apply machine learning or natural language processing techniques to provide specific answers to queries provided by the patient.

The disclosed embodiments are not limited to embodiments that provide interactive opportunities using treatment pathways. In some embodiments, patient competencies can be tracked and used to select interactive opportunities. Patient competencies can concern physical behaviors of a patient (e.g., activity level, sleep management, nutrition management, hydration management, medication adherence, or the like); mental behaviors of the patient (e.g., stress management, or psychological management, or the like); knowledge about the patient's condition or relevant treatments; patient characteristics (e.g., pain level, range of motion, or the like); or other similar competencies. Such a competency-based approach may be more flexible (though less-structured) than a treatment pathway-based approach. In some embodiments, target competencies can be expressly or implicitly maintained. Each of the target competencies can be specific to a patient, specific to patients satisfying one or more criteria (e.g., patients having the same diagnosis; similar patient information as described herein, or the same combination of diagnosis and patient information, or other criteria relevant to target competency levels of the patient), or the same for all patients. Selection of interactive opportunities can then depend on the tracked and target competency values for a patient. In some embodiments, a weight can be associated with each competency. Each weight can be specific to a patient, specific to patients satisfying one or more criteria (e.g., patients having the same diagnosis; similar patient information as described herein, or the same combination of diagnosis and patient information, or other criteria relevant to competency weights for the patient), or the same for all patients. Selection of interactive opportunities can then depend on these weights, in addition to the tracked and target competency values for a patient.

In some embodiments, a multi-level recommendation architecture can be used to select interactive opportunities for a patient. A first level of the recommendation architecture can generate competency-specific lists of interactive opportunities. Each competency-specific list can be selected by selecting interactive opportunities from an overall pool (or pools) of interactive opportunities. Each competency-specific list can be associated with a particular competency and can include the most highly recommended interactive opportunities for that competency. A second level of the recommendation architecture can generate a patient-specific list of interactive opportunities. The patient-specific list can be generated by selecting interactive opportunities from the competency-specific lists. In various embodiments, the number of interactive opportunities selected from each of the competency-specific lists (and optionally the ordering of the interactive opportunities within the patient-specific list) can be specific to a patient, specific to patients satisfying a criterion, or the same for all patients. For example, a diagnosis can be associated with a set of weights. When a patient profile indicates that diagnosis, the weights associated with that diagnosis can be used to generate the patient-specific list of interactive opportunities.

The multi-level recommendation architecture can be updated based on feedback obtained from a patient. In some embodiments, such feedback can include patient information (e.g., as described with regards to patient information 1521) or patient interaction data (e.g., as described with regards to patient interaction data 1523). In some embodiments, feedback can differently affect the different levels of the multi-level recommendation architecture. For example, feedback indicating selection of an opportunity by a patient may increase the likelihood of selecting that opportunity for a competency-specific list (as a patient was selected, and was therefore interested in, that opportunity). Such feedback may also decrease the likelihood of subsequently selecting that opportunity for a patient-specific list for that particular patient (as they have already performed that interactive opportunity).

In this manner, the first level of the recommendation architecture can be tuned to select the most relevant interactive opportunities for patients in general, while second level of the multi-level architecture can ensure that the interactive opportunities presented to a particular patient are personalized to that patient.

As may be appreciated, the multi-level recommendation architecture can be combined with aspects of other disclosed embodiments. For example, the views and functionality described with regards to FIGS. 4, 5A, 5B, 6, 7, 9, 10, 11A-11D, 12, 13 and 14A to 14C can be used in conjunction with interactive opportunities recommended using the disclosed multi-level recommendation architecture. Similarly, the disclosed multi-level recommendation architecture can be implemented within the game loop described with regards to FIG. 8. In some embodiments, for example, interactive opportunities 803 can be recommended, at least in part, using the disclosed multi-level recommendation architecture.

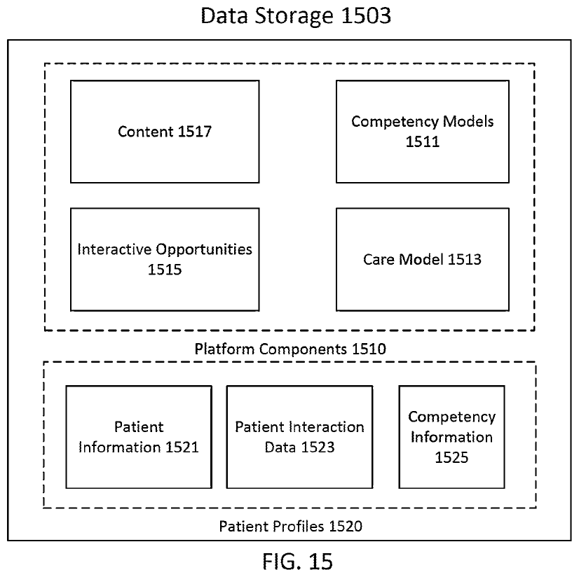

FIG. 15 depicts exemplary contents of data storage 1503 of the architecture of FIG. 1, in accordance with disclosed embodiments. The depiction in FIG. 15 is a logical depiction: the depicted contents of data storage 1503 can be distributed across multiple physical machines (including, in some embodiments, patients' system(s) 105). The depicted contents can include platform components 1510 and patient profiles 1520. Platform components 1510 can include objects or data useable with multiple patients, while patient profiles 1520 can include objects or data associated with a particular patient. The disclosed embodiments are not limited to any particular implementation or format for platform components 1510 or patient profiles 1520.

Similar to platform components 210, described above with regards to FIG. 2, platform components 1510 can include interactive opportunities 1515 and content 1517. Interactive opportunities 1515 can include data or instructions for implementing missions, questionnaires, data submissions, patient communications, or the like (e.g., interactive opportunities). Interactive opportunities 1515 can form a pool of interactive opportunities that competency models (or in some embodiments, a care model) can select from, as described herein.

In some embodiments, an interactive opportunity can include competency characteristics, preconditions, or postconditions. Consistent with disclosed embodiments, competency characteristics can include associations between the interactive opportunity and one or more patient competencies. For example, a mission in which the patient must walk a certain distance in a certain time can be associated with an activity level competency, as well as a stress management competency (as exercise can reduce stress) and a sleep management competency (as exercise can improve sleep). In some embodiments, a strength of associations between the interactive opportunity and each of the one or more patient competencies can differ (e.g., a walking mission can be more strongly associated with an activity level competency than with a stress management competency, and more strongly associated with the stress management competency than with a sleep management competency). These differing strengths can reflect an assessment (e.g., by a creator or reviewer of the interactive opportunity) of the strength of a causal relationship between performance of the activity and improvement in the associated patient competency.

Consistent with disclosed embodiments, preconditions can include criteria that must be satisfied in order to select an interactive opportunity for inclusion in a list (e.g., a competency-specific list or the patient-specific list). Such criteria can include a minimum patient competency level in one or more competencies, which can be selected as relating to successful performance of the interactive opportunity. For example, an interactive opportunity requiring the patient complete a 500-yard swim may include a minimum activity level, pain level, range of motion, or medication adherence competency value, as each such competency may affect the ability of the patient to complete (or to safely complete) the interactive opportunity. Additionally or alternatively, such criteria can include a maximum patient competency level in one or more competencies. A maximum competency level can ensure that selected interactive opportunities are suitably challenging for a patient. For example, an interactive opportunity to watch a video describing basic facts of a medical condition may specify a low maximum competency level in an education competency, so that this interactive opportunity is not presented to a patient already familiar with that medical condition. Additionally or alternatively, such criteria can include a range of patient competency levels in one or more competencies.

Consistent with disclosed embodiments, postconditions can include instructions on updating competencies associated with an interactive opportunity level. In some embodiments, an interactive opportunity may specify that a competency value be incremented to a value or by an amount upon successful completion of the opportunity. For example, a postcondition on a mission to run a marathon may include setting an activity competency of the patient to its maximum value. In some embodiments, an interactive opportunity may specify that a competency value be decremented to a value or by an amount upon failure to complete the opportunity. For example, a postcondition on an educational mission to answer a basic quiz about a medical condition may include setting an education competency of the patient to its minimum value. In some embodiments, multiple successful (or unsuccessful) missions may be required to increment (or decrement) a competency value. A postcondition can specify which competencies are incremented (or decremented) and how much the mission counts towards incrementing or decrementing these competencies.

Content 1517 can include interface content used by the patient support platform interface (e.g., icons, interface graphics, sounds, or the like), educational content (e.g., concerning diseases or conditions, treatments, or the like), questionnaires, or the like. In some embodiments, content 1517 can include graphical, audio, or audiovisual content, or other suitable content. In some embodiments, interactive opportunities 1515 can include instructions for referencing items of content 1517.

Consistent with disclosed embodiments, platform components 1510 can include competency models 1511 and care model 1513. In some embodiments, when care model 1513 is personalized to a patient, patient profiles 220 can include patient-specific care models. Competency models 1511 can include competency models for selecting interactive opportunities for inclusion in a competency-specific list. Consistent with disclosed embodiments, each competency model can be associated with a patient competency. Inputs to a competency model can include information concerning interaction opportunities; competency information for the patient; patient information; patient interaction data, or the like. Outputs to a competency model can include a ranking of the interaction opportunities, or a selection (optionally a ranked selection) of the interaction opportunities.

As may be appreciated, the outputs of each competency model can be independent of the output of the remaining competency models. Using independent models can enable additional competencies and competency-specific models to be conveniently added to the patient support platform without having to modify or adjust the remaining competency models. However, as a result of this independence, the same mission may be chosen for multiple competency-specific lists. In such instances, the patient support platform (e.g., the care model or another component of the patient support platform) can be configured to de-duplicate the competency-specific lists.

In some embodiments, competency models can be trained based on feedback provided by patients using the patient support platform. For example, a competency model can select an interactive opportunity, that interactive opportunity can be presented to the patient, and the patient can select (and optionally successfully perform) that interactive opportunity. The competency model can subsequently be updated, modified, trained, or the like to become more likely to select that interactive opportunity. If the patient does not select the interactive opportunity (or optionally does not successfully perform the interactive opportunity) the competency model can subsequently be updated, modified, trained, or the like to become less likely to select that interactive opportunity.

The disclosed embodiments are not limited to a particular implementation of the competency models. In some embodiments, the competency models can be or include one or more regression models, decision tree models, support vector models, k-nearest neighbor models, neural network models, ensemble models, or other machine learning models.

Care model 1513 can include a care model for selecting interactive opportunities from competency specific lists for inclusion in a patient-specific list. In some embodiments, the interactive opportunities can be selected from a pool of opportunities. The pool of opportunities can include the interactive opportunities stored on the patient support platform (e.g., interactive opportunities 1515, or the like).

In some embodiments, a care model (e.g., care model 1513) can be specific to a patient. In various embodiments, the care model may not be specific to a patient. For example, a single care model can be configured to generate patient-specific lists for multiple patients. Inputs to a care model can include information concerning interaction opportunities; competency information for the patient; target competency information for the patient; competency weights; patient information; patient interaction data, or the like. For example, an indication of a diagnosis can be provided to the care model. The care model can be configured to determine competency weights for generating a patient-specific list based on the indicated diagnosis. Outputs to a care model can include a ranking of the interaction opportunities (e.g., of the interaction opportunities in the competency-specific lists, of the interaction opportunities in data storage 103, or the like), or a selection (optionally a ranked selection) of the interaction opportunities. In some embodiments, selecting opportunities from competency-specific lists for inclusion in a patient-specific list can include de-duplicating the interactive opportunities.

In some embodiments, the competency-specific lists can be ranked lists. The care model (e.g., care model 1513) can be configured to select interactive opportunities from each ranked list in rank order. For example, the first interactive opportunity selected from a competency-specific list can be the highest-ranked interactive opportunity in that competency-specific list, the second interactive opportunity selected from the competency-specific list can be the second-highest-ranked interactive opportunity in that competency-specific list, etc. In some embodiments, the competency-specific lists can be unranked lists. The care model can be configured to select interactive opportunities from each list in random order or according to a ranking or selection criterion applied by the care model.

Consistent with disclosed embodiments, the care model can select interactive opportunities from competency lists according to constraints. In some embodiments, the constraints can specify a number of interactive opportunities that can be provided to a patient system in a specified period of time (e.g., per day, per week, or the like). In some embodiments, the constraints can impose compatibility conditions on the interactive opportunities. In some implementations, duplicate interactive opportunities or interactive opportunities that are too similar may be deemed incompatible. Two interactive opportunities that require substantial physical exertion (e.g., a mission to run a mile and a mission to swim 500 yards) may be deemed too similar and thus incompatible. Likewise, two interactive opportunities that require watching video content and answering questions (e.g., a mission to listen to a video describing a medical diagnosis and take a follow-up quiz, and a mission to listen to a video describing proper nutrition concepts and take a follow-up quiz) may be deemed too similar and thus incompatible. Likewise, two interactive opportunities that overlap may be deemed incompatible. Two missions can overlap when performance of once mission would constitute performance of the other mission. For example, a mission to run a mile can overlap with a mission to run two miles. Similarly, a mission to eat a serving of leafy greens for lunch can overlap with a mission to eat two servings of vegetables for lunch.

The disclosed embodiments are not limited to a particular implementation of the care model. In some embodiments, the care model can be or include one or more regression models, decision tree models, support vector models, k-nearest neighbor models, neural network models, ensemble models, or other machine learning models.

Similar to patient profiles 220, described above with regards to FIG. 2, patient profiles 1520 can include patient information 1521 and patient interaction data 1523. Similar to patient information 221, patient information 1521 can include patient medical information, demographic information, location information, or the like. Patient information 221 can include information obtained from the patient, information obtained from another party regarding the patient, a medical record of the patient, activity data obtained from a device associated with the patient, or the like. Similar to patient interaction data 223, patient interaction data 1523 can include a record of the interactive opportunities selected by the patient, customization options selected by the patient for the selected interactive opportunities, whether the selected interactive opportunities were completed, data pertaining to the performance of the selected interactive opportunities, communications between the patient and a coach, amount of time logged into the platform, or other suitable interactions with the patient support platform.

Patient profiles 1520 can include competency information 1525. In various embodiments, competency information 1521 can include patient competency levels or target competency levels. The disclosed embodiments are not limited to any particular implementation of target competency levels or patient competency levels. In some embodiments, a patient competency level or target competency level can be a level or category (e.g., a number between a minimum and maximum value, an element in an enumeration, or the like) or a value (e.g., average number of minutes spent in physical activity in a week, average number of steps in a day, score on most recent educational questionnaire, Body Mass Index, hours slept in last week, or the like). The patient competency level or target competency level can be expressly (e.g., as a value in a key-value pair, a field value in an object, or the like) or implicitly (e.g., through position in an ordered list or matrix of values, or the like) associated with a patient competency.

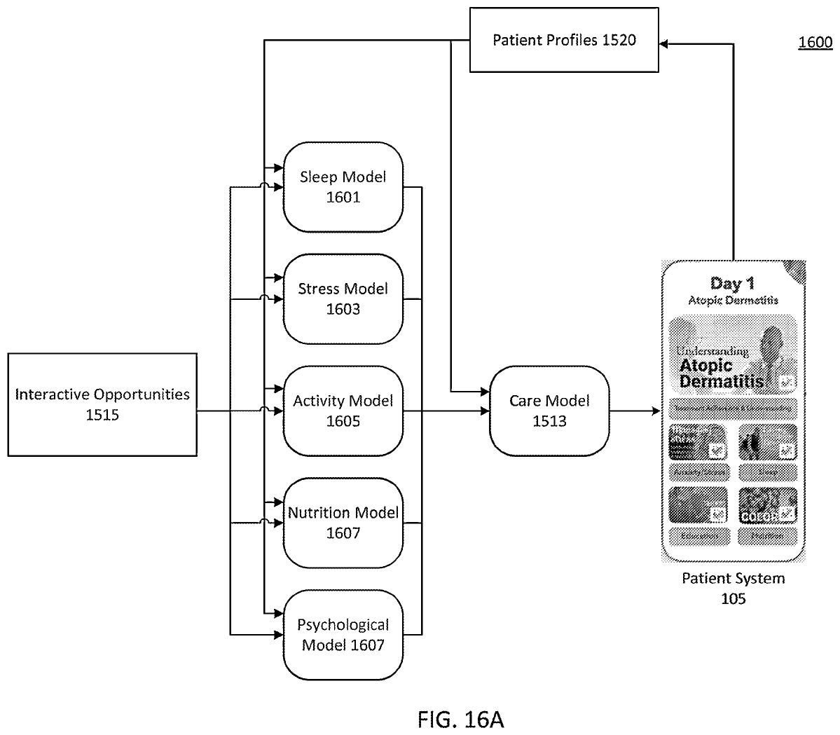

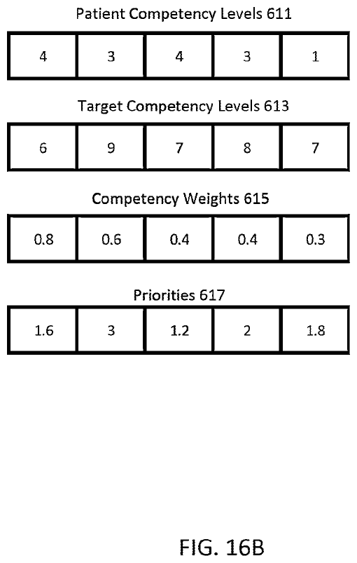

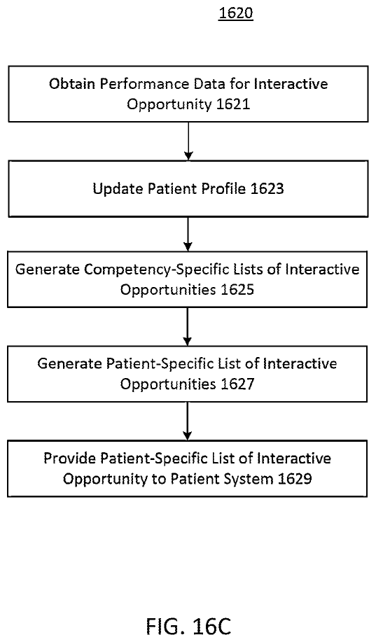

FIG. 16A depicts an exemplary architecture 1600 for selecting personalized interactive opportunities, in accordance with disclosed embodiments. Architecture 1600 includes a selection of competency models (e.g., sleep model 1601, stress model 1603, activity model 1605, nutrition model 1607) and a care model 1513. Each of the competency models can be configured to generate competency-specific lists by selecting ones of interactive opportunities 1515.

In some embodiments, information from patent profiles 1520 can be used by the competency models in generating the competency-specific lists. In some embodiments, such information can include patient interaction data (e.g., items of patient interaction data 1523, or the like). For example, such patient interaction data can include previously selected interactive opportunities (and whether the patient completed the selected interactive opportunities successfully). In some embodiments, the competency models can be configured not to select interactive opportunities previously presented to the patient (or optionally previously presented and selected). As an additional example, such patient interaction data can include how the patient performed on previously selected interactive opportunities. Such interaction data can include activity or biometric data acquired by a wearable device of the patient, such as step or heartrate data. Additionally or alternatively, such interaction data can include results of a patient questionnaire, or the like.

In some embodiments, such information can include patient competency information (e.g., items of patient competency information 1525). The patent competency information can be used to determine whether preconditions are satisfied for interactive opportunities. As described herein, satisfaction of a precondition may require that a patient possesses a competency level greater than a minimum competency level (or less than a maximum competency level).

Consistent with disclosed embodiments, the competency-specific lists of interactive opportunities can be input to care model 1513. Care model 1513 can be configured to generate a patient-specific list of interactive opportunities by selecting from among the interactive opportunities on the competency-specific lists. In some embodiments, information from patent profiles 1520 can be used by care model 1513 in generating the competency-specific lists. In some embodiments, such information can include patient interaction data (e.g., items of patient interaction data 1523, or the like). Such patient interaction data can include the interactive opportunities previously selected (or selected and successfully completed) by the patient. Care model 1513 can use such information to ensure that previously selected interactive opportunities are not repeatedly provided to the patient. In some embodiments, such information can include patient information (e.g., items of patient information 1521). Such items can include an indication of a diagnosis of the patient, which can be used to determine competency weights. In some embodiments, such information can include competency information (e.g., items of competency information 1525). As described below with regards to FIG. 16B, the patient competency information can be used to determine priorities for selecting interactive opportunities from the competency-specific lists generated by the competency models. In some embodiments, care model 1513 can select interactive opportunities for inclusion into the patient-specific list based on these priorities.

Consistent with disclosed embodiments, interactive opportunities can be provided to patient system 105 according to the patient-specific list. In some embodiments, the list of interactive opportunities can be provided to patient system 105. In response to a selection of one of the interactive opportunities, necessary data and instructions for performing the interactive opportunity can be provided. In various embodiments, data and instructions for performing all of the interactive opportunities can be provided to patient system 105.

Consistent with disclosed embodiments, the patient can select one of the provided interactive opportunities. In some instances, the patient can successfully (or unsuccessfully) perform this interactive opportunity. In some embodiments, the patient may have the opportunity to suspend or abort performance of the interactive opportunity (and may select another opportunity). The patient support platform can obtain performance data for the interactive opportunity. As described herein with respect to FIG. 3, such performance data can be push to or retrieved by the patient support platform. The specific performance data obtained can depend on the interactive opportunity performed. The disclosed embodiments are not limited to any particular type or format of performance data. In various embodiments, the performance data can include an indication that an interactive opportunity was completed (or not completed, or suspected or aborted) by the patient; data received from the patient system; data obtained by a wearable device of the patient; or the like. In some embodiments, as described herein, the patient support platform can communicate with the patient system regarding the interactive opportunity.

As described herein with respect to FIG. 3, the patient support platform can update competency information for the patient (e.g., competency information 1525) based on the obtained performance data. The performance data can implicate multiple competencies tracked by the patient support system for the patient. In some instances, the patient support platform can be configured to update competency information once an indication has been received that performance of an interactive opportunity is complete.

In some embodiments, a competency level of the patient can be updated according to postconditions associated with the interactive opportunity. For example, a postcondition on an exercise interactive opportunity can specify that successfully completion of the exercise interactive opportunity result in a one-unit increment of the patient's activity competency level and a one-unit increment of the patient's sleep competency level (e.g., according to the rationale that exercise improves physical condition and sleep hygiene). In some embodiments, the postcondition can specify that partial completion of the exercise interactive opportunity result in no increment (or even a decrement) of the patient's activity competency level and a one-unit increment of the patient's sleep competency level (e.g., according to the rationale that performing even a limited amount of exercise retains a beneficial effect on sleep).

Consistent with disclosed embodiments, the patient support platform can update patient interaction data for the patient (e.g., patient interaction data 1523) based on the obtained performance data. In some embodiments, the updates can indicate which interactive opportunity was selected (and optionally which interactive opportunities were presented, but not selected). In some embodiments, the updates can indicate selection or rejection of additional configuration opportunities within the interactive opportunity (e.g., selection of a distance to run, answers to questions in a questionnaire, pausing or fast-forwarding an informational video, or the like).

Consistent with disclosed embodiments, the patient support platform can update patient information for the patient (e.g., patient information 1521) based on the obtained performance data. The patient information can be updated using information obtained from patient system 105 or from a wearable device associated with the patient. For example, the patient information can be updated to store a number of steps taken (or distance traveled, or the like) during performance of an exercise interactive opportunity. As an additional example, the patient information can be updated to store an image of the meal of a patient taken during a nutrition counselling interactive opportunity. As a further example, the patient information can be updated to store the heart rate of the patient during performance of a meditation interactive opportunity.

As may be appreciated, the updated patient profile can be used by the patient support system in determining subsequent patient-specific lists of interactive opportunities. For example, the competency modules can use the updated patient profile when compiling competency-specific lists. In some instance, due to increases in competency levels, new interactive opportunities may be available to a patient. For example, an educational competency of the patient may increase following successful completion of an introductory educational interactive opportunity. The increased level of the educational competency may satisfy a precondition on a more advanced educational interactive opportunity. An educational competency module may therefore select this more advanced educational interactive opportunity for subsequent inclusion in a competency-specific list.

Consistent with disclosed embodiments, the patient support platform can be configured to repeatedly generate new patient-specific lists. In some embodiments, the patient-specific lists can be generated periodically (e.g., every day, every week, or the like), according to a schedule (e.g., Tuesday and Thursday every week, or the like), or the like. In various embodiments, the patient-specific lists can be generated in response to an event (e.g., successful or unsuccessful completion of a selected interactive opportunity, successful or unsuccessful completion of all interactive opportunities in the most-recently provided patient specific list, a change in the patient competency levels for the patient, a request for new interactive opportunities received from the patient system, passage of a predetermined amount of time from the last time the patient selected an interactive opportunity, or other events concerning the patient or patient interactions with the patient support system).

FIG. 16B depicts exemplary inputs to a care model (e.g., care model 1513) used to select personalized interactive opportunities, in accordance with disclosed embodiments. In some embodiments, personalized interactive opportunities can be selected according to priorities (e.g., priorities 617). These priorities can be determined based on patient competency levels (e.g., patient competency levels 611) and target competency levels (e.g., target competency levels 613). In some embodiments, the priorities can further depend on competency weights (e.g., competency weights 615). In some embodiments, competency weights can depend on patient information (e.g., items of patient information 1521). For example, competency weights can be associated with diagnosis. Patient information can include an indication of that diagnosis, which can be used to obtain an appropriate set of competency weights. Consistent with disclosed embodiments, the patient competency levels, target competency levels, and/or competency weights can be stored in competency information 1525, or a similar location.

Consistent with disclosed embodiments, a patient competency level or target competency level can be a level or category or a value. The patient competency level or target competency level can be expressly or implicitly associated with a patient competency.

Consistent with disclosed embodiments, FIG. 16B depicts patient competency levels 611 as being vector of numeric competency levels. In this example, the association between each level and the corresponding competency is implicit in the ordering of the levels within the vector. The implicit order is sleep competency (level 4), stress competency (level 3), activity competency (level 4), nutrition competency (level 3), and psychological competency (level 1). As may be appreciated, the disclosed embodiments are not limited to such an implementation.

Consistent with disclosed embodiments, FIG. 16B depicts target competency levels 613 as being vector of numeric competency levels, similar in implementation to patient competency levels 611. As described herein, each of the target competency levels can be specific to the patient, specific to patients satisfying a criterion, or the same for all patients. In this example, the target sleep competency level is 6, the target stress competency level is 9, the target activity competency level is 7, the target nutrition competency level is 8, and the target psychological competency level is 7.

Consistent with disclosed embodiments, FIG. 16B depicts weights 615 as being vector of numeric values, each implicitly associated with a patient competency. As described herein, each of the weights can be specific to the patient, specific to patients satisfying a criterion, or the same for all patients. In this example, the sleep competency weight is 0.8, the stress competency weight is 0.6, the activity competency weight is 0.4, the nutrition competency weight is 0.4, and the psychological competency weight is 0.3.

Consistent with disclosed embodiments, priorities can be numeric or categorical values. Each priority can be associated with one or more competency. For example, FIG. 16B depicts priorities 617 as being vector of numeric values, each implicitly associated with a patient competency. In various embodiments, the priorities can be a function of the patient competency levels; a function of the patient competency levels and the target competency levels; or a function of the patient competency levels, target competency levels, and weights. In some embodiments, the priorities can also be a function of additional information, such as performance data regarding previously performed interactive opportunities, or patient profile information.

In the example depicted in FIG. 16B, the priorities are the product of the weight and the difference between the target competency level and the patient competency level for each competency: the priority of the sleep competency is 1.6, the priority of the stress competency is 3, the priority of the activity competency is 1.2, the priority of the nutrition competency is 2, and the priority of the psychological competency is 1.8.

Consistent with disclosed embodiments, the patient support platform can be configured with a mapping from priority values to selection of interactive opportunities. Numerous mappings are possible, and the disclosed embodiments are not restricted to any particular such mapping.

In some embodiments, a mapping can convert priorities into selection probabilities (e.g., using a softmax function, or the like). The care model can select interactive opportunities from the competency-specific lists according to these probabilities. For example, the priorities depicted in FIG. 16B can be converted to the probabilities 12%, 48%, 8%, 18%, and 14% using a softmax function. The care model could then select about 50% of the interactive opportunities from the stress competency-specific list, select about 20% from the nutrition competency-specific list, and select about 30% of the interactive opportunities approximately equally from the remaining competency specific lists.

In some embodiments, a mapping can convert priorities into a number of selections from each competency-specific list (e.g., through categorization of priority values). From each competency-specific list, the determined number of selections can be made. As a first example, the priorities depicted in FIG. 16B can be rounded up to the nearest whole number (e.g., 2, 3, 2, 2, 2). The care model can then select that number of interactive opportunities from each competency-specific list. For example, the care model can select three interactive opportunities from the stress competency-specific list and two interactive opportunities from each of the remaining four competency-specific lists. As a second example, the priorities depicted in FIG. 16B can be converted into high (priority greater than 2), medium (priority greater than 1.5 and less than 2), and low (priority less than 1.5) importance categories. The care model can select four interactive opportunities from competency-specific lists for high-importance competencies, two interactive opportunities from competency-specific lists for medium-importance competencies, and one interactive opportunity from competency-specific lists for low-importance competencies.

FIG. 16C depicts an exemplary method 1620 for selecting personalized interactive opportunities, in accordance with disclosed embodiments. Method 1620 can include operations of obtaining performance data for an interactive opportunity, updating a patient profile, generating competency-specific lists of interactive opportunities, generating a patient specific list of interactive opportunities, and providing the patient specific list to the patient system.

In step 1621, the patient support system can obtain performance data for an interactive opportunity selected by the patient, in accordance with disclosed embodiments. Consistent with disclosed embodiments, the patient may have selected the interactive opportunity from a patient-specific list of interactive opportunities selected by the patient support platform and provided to a patient system, as disclosed herein. The performance data can concern performance of the first interactive opportunity by the patient. For example, the performance data can indicate successful completion of the interactive opportunity by the patient. In some embodiments, the performance data can include activity or biometric data acquired by a wearable device of the user.

In step 1623, the patient support system can update a patient profile of the patient, in accordance with disclosed embodiments. Updating the patient profile can include updating patient information, patient interaction data, or patient competency information based on the obtained performance data. For example, when the performance data indicates successful completion of the interactive opportunity, updating the patient profile can include increasing at least one competency level associated with the interactive opportunity. In some embodiments, the interactive opportunity can have postconditions. The competency levels can be updated according to the postconditions of the interactive opportunity.

In step 1625, the patient support system can generate competency-specific lists by selecting among interactive opportunities (e.g., selecting among interactive opportunities 1515), in accordance with disclosed embodiments. The patient support system can use competency models to generate corresponding competency-specific lists. The competency models can select interactive opportunities based on the patient profile (e.g., based on patient information, patient interaction data, and competency information for the patient) or characteristics of the interactive opportunities (e.g., a strength of an association between an interactive opportunity and the competency, preconditions on selecting the interactive opportunity, or the like).

In step 1627, the patient support system can generate a patient-specific list of interactive opportunities, in accordance with disclosed embodiments. The patient support system can generate the patient-specific list by selecting among interactive opportunities included in the competency-specific lists. The patient can select the interactive opportunities for inclusion in the patient-specific lists using a care model. In some embodiments, the care model can be specific to the patient. In various embodiments, the care model can be used for multiple patients (e.g., all patients on the patient support platform, all patients on the patient support platform that satisfy a criterion, or the like). In some such embodiments, an indication of the patient or of a diagnosis of the patient can be input to the care model to personalize the care model to the patient.

As described herein, with regards to FIG. 16B, priorities can be associated with each of the competencies. The care model can select from among the competency-specific lists according to these priorities. In some embodiments, the priorities can be determined based on patient competency levels; patient competency levels and target competency levels; patient competency levels and competency weights; or patient competency levels, target competency levels, and competency weights. In some embodiments, the weights can be specific to the patient, a group of patients satisfying a criterion (e.g., a group of patients having the same diagnosis), or all patients on the patient support platform. In some embodiments, a priority can be the product of a competency weight and a difference between a target competency level and a corresponding patient competency level. In some embodiments, priorities can be converted into selection probabilities for selecting interactive opportunities from each of the competency-specific lists. In various embodiments, priorities can be converted into selection numbers for selecting interactive opportunities from each of the competency-specific lists.

In step 1629, the patient support system can provide the patient-specific list to the patient system. As described herein, in some embodiments, the patient-specific list can be provided to the patient system. In response to a selection of one of the interactive opportunities, necessary data and instructions for performing the selected interactive opportunity can be provided. In various embodiments, data and instructions for performing all of the interactive opportunities in the patient-specific list can be initially provided to patient system 105.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Embodiments herein include systems, methods, and tangible non-transitory computer-readable media. The methods may be executed, at least in part for example, by at least one processor that receives instructions from a tangible non-transitory computer-readable storage medium. Similarly, systems consistent with the present disclosure may include at least one processor and memory, and the memory may be a tangible non-transitory computer-readable storage medium. As used herein, a tangible non-transitory computer-readable storage medium refers to any type of physical memory on which information or data readable by at least one processor may be stored. Examples include random access memory (RAM), read-only memory (ROM), volatile memory, non-volatile memory, hard drives, CD ROMs, DVDs, flash drives, disks, registers, caches, and any other known physical storage medium. Singular terms, such as “memory” and “computer-readable storage medium,” may additionally refer to multiple structures, such a plurality of memories or computer-readable storage media. As referred to herein, a “memory” may comprise any type of computer-readable storage medium unless otherwise specified. A computer-readable storage medium may store instructions for execution by at least one processor, including instructions for causing the processor to perform steps or stages consistent with embodiments herein. Additionally, one or more computer-readable storage media may be utilized in implementing a computer-implemented method. The term “non-transitory computer-readable storage medium” should be understood to include tangible items and exclude carrier waves and transient signals.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure. Therefore, it is intended that the disclosed embodiments and examples be considered as examples only, with a true scope of the present disclosure being indicated by the following claims and their equivalents.

The embodiments may further be described using the following clauses:

1. A treatment support system, comprising: at least one processor; and at least one computer-readable medium containing instructions that, when executed by the at least one processor, cause the treatment support system to perform operations comprising: provide a first mission to a client associated with a user, the user associated with a first treatment pathway; receive mission data from the client, the mission data concerning performance of the first mission by the user; based on the received mission data, associate the user with a second treatment pathway; select second missions for provision to the user based, in part, on the association of the user with the second treatment pathway; and provide an indication of the second missions to the client.

2. The treatment support system of clause 1, wherein: the second treatment pathway comprises a sequence of modules; associating the user with the second treatment pathway comprises associating the user with a first module in the sequence of modules, the first module associated with a first set of missions; and selecting the second missions comprises selecting the second missions from the first set of missions associated with the first module.

3. The treatment support system of any one of clauses 1 to 2, wherein: the operations further include: determining that the user has satisfied a mastery condition associated with the first module; associating the user with a second module of the sequence of modules, the second module associated with a second set of missions; and providing an indication of the second missions to the client.

4. The treatment support system of clause 3, wherein: the first set of missions includes at least one mandatory mission; and satisfaction of the mastery condition depends, at least in part, upon performance of the at least one mandatory mission.

5. The treatment support system of any one of clauses 2 to 4, wherein: the first set of missions includes a sequence of missions; and the selection of the second missions depends, in part, on a position of the user in the sequence of missions.

6. The treatment support system of any one of clauses 1 to 5, wherein: selecting the second missions comprises selecting missions associated with the first treatment pathway and missions associated with the second treatment pathway.

7. The treatment support system of clause 6, wherein: a value of a skill is associated with the user; the second treatment pathway concerns development of the skill; the operations further comprise determining, based on the received mission data, that the user requires development of the skill; and the user is associated with the second treatment pathway in response to the determination.

8. The treatment support system of any one of clauses 1 to 7, wherein: the missions associated with the first treatment pathway are each associated with a point value; the first treatment pathway is associated with a point total; and the missions associated with the first treatment pathway are selected based on the point total and the point values.

9. The treatment support system of any one of clauses 1 to 8, wherein: providing the first mission to the client comprises configuring the first mission based on stored data concerning missions previously competed by the user.

10. The treatment support system of any one of clauses 1 to 9, wherein: the selection of the second missions depends upon stored data concerning: missions previously competed by the user; or values of skills associated with the user.

11. The treatment support system of any one of clauses 1 to 10, wherein: the operations further comprise: receiving an identifier from the user; and based on the identifier, associating the user with the first treatment path.

12. The treatment support system of any one of clauses 1 to 11, wherein: the mission data includes activity or biometric data acquired by a wearable device of the user.

13. A treatment support method, comprising: associating a user with a first module on a first treatment pathway; selecting, at least in part from among missions associated with the first module on the first treatment pathway, first missions for the user to perform; providing indications of the first missions to the client; receiving a selection of one of the first missions from the client; providing the selected mission to the client; receiving mission data for the selected mission from the client; automatically determining that the user should be transferred to a second treatment pathway, based on the mission data; and associating the user with a first module on the second treatment pathway; selecting, at least in part from among missions associated with the first module on the second treatment pathway, second missions for the user to perform; and providing indications of the second missions to the client.

14. The treatment support system of clause 13, wherein the method further comprises: determining that the user has satisfied a mastery condition associated with the first module on the second treatment pathway; associating the user with a second module on the second treatment pathway; and selecting, from among missions associated with the second module on the second treatment pathway, third missions for the user to perform; and providing indications of the third missions to the client.

15. The treatment support system of clause 14, wherein: the second missions include at least one mandatory mission; and satisfaction of the mastery condition depends, at least in part, upon performance of the at least one mandatory mission.

16. The treatment support system of any one of clauses 13 to 15, wherein: the missions associated with the first module of the first treatment pathway are each associated with a point value; the first treatment pathway is associated with a point total; and the missions associated with the first module of the first treatment pathway are selected based on the point total and the point values.

17. The treatment support system of clause 16, wherein: a value of a skill is associated with the user; the second treatment pathway concerns development of the skill; the method further comprises determining, based on the received mission data, that the user requires development of the skill; and the user is associated with the a first module on the second treatment pathway in response to the determination.

18. The treatment support system of any one of clauses 13 to 17, wherein: the missions associated with the first module on the first treatment pathway include a sequence of missions; and the selection of the first missions depends, in part, on a position of the user in the sequence of missions.

19. The treatment support system of any one of clauses 13 to 18, wherein: selecting the second missions comprises selecting, at least in part, from among missions associated with the first module of the first treatment pathway and the first module of the second treatment pathway. 20. The treatment support system of any one of clauses 13 to 19, wherein: the selection of the second missions depends upon stored data concerning: missions previously competed by the user; or values of skills associated with the user.

21. The treatment support system of any one of clauses 13 to 20, wherein: the operations further comprise: receiving an identifier from the user; and based on the identifier, associating the user with the first treatment path.

22. The treatment support system of any one of clauses 13 to 21, wherein: providing the selected mission to the client comprises configuring the selected mission based on stored data concerning missions previously competed by the user.

23. The treatment support system of any one of clauses 13 to 22, wherein: the mission data includes activity or biometric data acquired by a wearable device of the user.

24. A treatment support method, comprising: associating a user with a first module on a first treatment pathway; providing missions to a client for the user to perform based on the first module on the first treatment pathway; receiving mission results from the client; associating, based on the mission results, the user with a first module on a second treatment pathway; and providing missions to the client for the user to perform based on the first module on the second treatment pathway.

25. A treatment support method for providing personalized treatment support using predetermined treatment pathways, comprising: positioning a patient at a first location, the first location being a first module on a first treatment pathway; selecting first treatment support missions for the patient based on the first location; providing at least one of the selected first treatment support missions to the patient; receiving mission data for the at least one of the selected treatment support missions; identifying, based on the received mission data, a secondary condition of the patient; repositioning the patient at a second location, the second location being a first module on a second treatment pathway; and providing at least one second treatment support mission for the patient based on the second location.

26. The treatment support method of clause 25, further comprising: repositioning the patient at the first location, following completion of the at least one second treatment support mission; and providing, after repositioning the patient at the first location, at least one third treatment support mission for the patient based on the first location.

27. A treatment support method, comprising: selecting, based on a position of a user at a first module on a first predetermined treatment pathway, among first missions associated with the first module; providing at least one of the selected first missions to a client for the user to perform; obtaining, from the client, first mission data concerning performance of the at least one of the selected first missions; updating the position of the user, based on the obtained first mission data, to a first module on a second predetermined treatment pathway; selecting among second missions associated with the first module on the second predetermined treatment pathway; and providing at least one of the selected second missions to the client.

28. The treatment support method of clause 27, wherein the method further comprises: obtaining, from the client, second mission data concerning the at least one of the selected second missions; determining that the user has satisfied a mastery condition; updating the position of the user, based on the determination, to a second module on the second predetermined treatment pathway; selecting among third missions associated with the second module on the second predetermined treatment pathway; and providing at least one of the selected third missions to the client.

29. The treatment support method of clause 28, wherein: the selected second missions include at least one mandatory mission; and satisfaction of the mastery condition depends, at least in part, upon performance of the at least one mandatory mission.

30. The treatment support method of any one of clauses 27 to 29, wherein: the second missions include a mission sequence; and the selection of the second missions depends, in part, on a position of the user in the mission sequence.

31. The treatment support method of any one of clauses 27 to 30, wherein: a value of a skill is associated with the user; the second treatment pathway concerns development of the skill; the method further comprises determining, based on the first received mission data, that the user requires development of the skill; and the position of the user is updated to the first module on the second treatment pathway in response to the determination.

32. The treatment support method of any one of clauses 27 to 31, wherein: the method further comprises: reselecting among first missions associated with the first module on the first predetermined treatment pathway; and providing, together with the at least one of the selected second missions, at least one of the reselected first missions.

33. The treatment support method of any one of clauses 27 to 32, wherein: the first missions are each associated with a point value; the first treatment pathway is associated with a point total; and the reselection among the first missions is based on based on the point total and the point values.

34. The treatment support method of any one of clauses 27 to 33, wherein: providing the at least one of the selected first missions comprises configuring the at least one of the selected first missions based on stored data concerning missions previously competed by the user.

35. The treatment support method of any one of clauses 27 to 34, wherein: the selection of the second missions depends upon stored data concerning: missions previously competed by the user; or values of skills associated with the user.

36. The treatment support method of any one of clauses 27 to 35, wherein: the operations further comprise: receiving an identifier from the user; and based on the identifier, associating the user with the first treatment path.

37. The treatment support method of any one of clauses 27 to 36, wherein: the first mission data includes activity or biometric data acquired by a wearable device of the user.

38. A treatment support system comprising: At least one processor; and At least one non-transitory computer-readable medium containing instructions that, when executed by the at least one processor, cause the treatment support system to perform the treatment support method of any one of clauses 24 to 37.

39. A treatment support system, comprising: at least one processor; and at least one computer-readable medium containing instructions that, when executed by the at least one processor, cause the treatment support system to perform operations comprising: provide a patient-specific list including a first interactive opportunity to a patient system associated with a patient; obtain performance data from the patient system, the performance data concerning performance of the first interactive opportunity by the patient; update a patient profile of the patient based on the obtained performance data; generate a first competency-specific list by selecting among interactive opportunities according to a competency model and the patient profile; generate a second patient-specific list of interactive opportunities by selecting among interactive opportunities included in the first competency-specific list and in at least one second competency-specific list according to a care model and the patient profile; and provide the second patient-specific list to the patient system.

40. The treatment support system of clause 39, wherein: the patient profile includes patient competency levels and corresponding target competency levels, and the care model is configured to select among the interactive opportunities included in the first competency-specific list and in the at least one second competency-specific list based on differences between the target competency levels and the corresponding patient competency levels.

41. The treatment support system of any one of clauses 39 to 40, wherein: the patient profile includes at least one patient competency level, and the care model is configured to select among the interactive opportunities based on the at least one patient competency level.

42. The treatment support system of clause 41, wherein: the obtained performance data indicates successful completion of the first interactive opportunity; and updating the patient profile based on the obtained performance data comprises increasing the at least one patient competency level.

43. The treatment support system of any one of clauses 39 to 42, wherein: the patient profile includes an indication of a diagnosis of the patient; and the care model is configured to select among the interactive opportunities included in the first competency-specific list and in the at least one second competency-specific list based on weights corresponding to the diagnosis.

44. The treatment support system of any one of clauses 39 to 43, wherein: the patient profile includes patient interaction data; and the competency model is configured to select among the interactive opportunities based on the patient interaction data.

45. The treatment support system of any one of clauses 39 to 44, wherein: the care model selects interactive opportunities from the first competency-specific list and the at least one second competency-specific list according to constraints, the constraints including: a number of interactive opportunities per day; and satisfaction of a compatibility criterion for the selected interactive opportunities.

46. The treatment support system of any one of clauses 39 to 45, wherein: the at least one second competency-specific list comprises multiple second competency-specific lists generated independently according to multiple second competency models and the patient profile.

47. The treatment support system of any one of clauses 39 to 46, wherein: the first competency-specific list and the at least one second competency-specific list include duplicate interactive opportunities; and generation of the second patient-specific list comprises deduplication of the duplicate interactive opportunities by the care model.

48. The treatment support system of any one of clauses 39 to 47, wherein: the competency model corresponds at least one of a sleep management competency, stress management competency, activity level competency, diet or nutrition management competency, hydration management competency, weight-management competency, treatment-engagement competency, pain management competency, and mental state or psychological management competency.

49. The treatment support system of any one of clauses 39 to 48, wherein: the performance data includes activity or biometric data acquired by a wearable device of the patient.

50. A treatment support system, comprising: at least one processor; and at least one computer-readable medium containing instructions that, when executed by the at least one processor, cause the treatment support system to perform operations comprising: generating first lists by independently selecting interactive opportunities from a pool of interactive opportunities using first models, the first models selecting the interactive opportunities from the pool based on: competency levels of a patient; and associations between the interactive opportunities and competencies of the patient; generating a second list by selecting interactive opportunities from the first lists using a second model, the second model selecting the interactive opportunities from the first lists based on: the competency levels of the patient; and target competency levels for the patient; providing the second list to a client device of the patient; receiving from the client device a selection of an interactive opportunity in the second list; providing the selected interactive opportunity to the client device; receiving performance data from the client device, the performance data concerning performance of the selected interactive opportunity by the patient; and updating the competency levels of the patient based on the received performance data.

51. The treatment support system of clause 50, wherein: the second model selects the interactive opportunities based on differences between the competency levels and corresponding ones of the target competency levels.

52. The treatment support system of any one of clauses 50 to 51, wherein: the received performance data indicates successful completion of the selected interactive opportunity; and updating a patient profile based on the received performance data comprises increasing values of competencies associated with the selected interactive opportunity.

53. The treatment support system of clause 52, wherein: the first models select the interactive opportunities from the pool based in part on the patient profile.

54. The treatment support system of any one of clauses 52 to 53, wherein: the first models select the interactive opportunities from the pool based in part on patient interaction data or patient information included in the patient profile.

55. The treatment support system of any one of clauses 50 to 54 wherein: the second model selects interactive opportunities from the first lists according to constraints, the constraints including: a number of interactive opportunities per day; and satisfaction of a compatibility criterion for the selected interactive opportunities.

56. The treatment support system of any one of clauses 50 to 55, wherein: the first lists include duplicate interactive opportunities; and generation of the second list comprises deduplication of the duplicate interactive opportunities by the second model.

57. The treatment support system of any one of clauses 50 to 56, wherein: the competencies of the patient include at least one of sleep management, stress management, activity level management, diet or nutrition management, hydration management, weight-management, treatment engagement, pain management, and mental state or psychological management.

58. The treatment support system of any one of clauses 50 to 57, wherein: the received performance data includes activity or biometric data acquired by a wearable device of the patient.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A treatment support system, comprising:
at least one processor; and
at least one computer-readable medium containing instructions that, when executed by the at least one processor, cause the treatment support system to perform operations comprising:
provide a first mission to a client associated with a user, the user associated with a first treatment pathway;
receive mission data from the client, the mission data concerning performance of the first mission by the user;
based on the received mission data, associate the user with a second treatment pathway;
select second missions for provision to the user based, in part, on the association of the user with the second treatment pathway; and
provide an indication of the second missions to the client.

2. The treatment support system of claim 1, wherein:
the second treatment pathway comprises a sequence of modules;
associating the user with the second treatment pathway comprises associating the user with a first module in the sequence of modules, the first module associated with a first set of missions; and
selecting the second missions comprises selecting the second missions from the first set of missions associated with the first module.

3. The treatment support system of claim 2, wherein:
the operations further include:
determining that the user has satisfied a mastery condition associated with the first module;
associating the user with a second module of the sequence of modules, the second module associated with a second set of missions; and
providing an indication of the second missions to the client.

4. The treatment support system of claim 3, wherein:
the first set of missions includes at least one mandatory mission; and
satisfaction of the mastery condition depends, at least in part, upon performance of the at least one mandatory mission.

5. The treatment support system of claim 2, wherein:
the first set of missions includes a sequence of missions; and
the selection of the second missions depends, in part, on a position of the user in the sequence of missions.

6. The treatment support system of claim 1, wherein:
selecting the second missions comprises selecting missions associated with the first treatment pathway and missions associated with the second treatment pathway.

7. The treatment support system of claim 6, wherein:
a value of a skill is associated with the user;
the second treatment pathway concerns development of the skill;
the operations further comprise determining, based on the received mission data, that the user requires development of the skill; and
the user is associated with the second treatment pathway in response to the determination.

8. The treatment support system of claim 1, wherein:
the missions associated with the first treatment pathway are each associated with a point value;
the first treatment pathway is associated with a point total; and
the missions associated with the first treatment pathway are selected based on the point total and the point values.

9. The treatment support system of claim 1, wherein:
providing the first mission to the client comprises configuring the first mission based on stored data concerning missions previously competed by the user.

10. The treatment support system of claim 1, wherein:
the selection of the second missions depends upon stored data concerning:
missions previously competed by the user; or
values of skills associated with the user.

11. The treatment support system of claim 1, wherein:
the operations further comprise:
receiving an identifier from the user; and
based on the identifier, associating the user with the first treatment path.

12. The treatment support system of claim 1, wherein:
the mission data includes activity or biometric data acquired by a wearable device of the user.

13. A treatment support method, comprising:
associating a user with a first module on a first treatment pathway;
selecting, at least in part from among missions associated with the first module on the first treatment pathway, first missions for the user to perform;
providing indications of the first missions to the client;
receiving a selection of one of the first missions from the client;
providing the selected mission to the client;
receiving mission data for the selected mission from the client;
automatically determining that the user should be transferred to a second treatment pathway, based on the mission data; and
associating the user with a first module on the second treatment pathway;
selecting, at least in part from among missions associated with the first module on the second treatment pathway, second missions for the user to perform; and
providing indications of the second missions to the client.

14. The treatment support system of claim 13, wherein the method further comprises:
determining that the user has satisfied a mastery condition associated with the first module on the second treatment pathway;
associating the user with a second module on the second treatment pathway; and
selecting, from among missions associated with the second module on the second treatment pathway, third missions for the user to perform; and
providing indications of the third missions to the client.

15. The treatment support system of claim 14, wherein:
the second missions include at least one mandatory mission; and
satisfaction of the mastery condition depends, at least in part, upon performance of the at least one mandatory mission.

16. The treatment support system of claim 13, wherein:
the missions associated with the first module of the first treatment pathway are each associated with a point value;
the first treatment pathway is associated with a point total; and
the missions associated with the first module of the first treatment pathway are selected based on the point total and the point values.

17. The treatment support system of claim 16, wherein:
a value of a skill is associated with the user;
the second treatment pathway concerns development of the skill;
the method further comprises determining, based on the received mission data, that the user requires development of the skill; and
the user is associated with the a first module on the second treatment pathway in response to the determination.

18. The treatment support system of claim 13, wherein:
the missions associated with the first module on the first treatment pathway include a sequence of missions; and
the selection of the first missions depends, in part, on a position of the user in the sequence of missions.

19. The treatment support system of claim 13, wherein:
selecting the second missions comprises selecting, at least in part, from among missions associated with the first module of the first treatment pathway and the first module of the second treatment pathway.

20. The treatment support system of claim 13, wherein:
the selection of the second missions depends upon stored data concerning:
missions previously competed by the user; or
values of skills associated with the user.

21. The treatment support system of claim 13, wherein:
the operations further comprise:
receiving an identifier from the user; and
based on the identifier, associating the user with the first treatment path.

22. The treatment support system of claim 13, wherein:
providing the selected mission to the client comprises configuring the selected mission based on stored data concerning missions previously competed by the user.

23. The treatment support system of claim 13, wherein:
the mission data includes activity or biometric data acquired by a wearable device of the user.

24. A treatment support method, comprising:
associating a user with a first module on a first treatment pathway;
providing missions to a client for the user to perform based on the first module on the first treatment pathway;
receiving mission results from the client;
associating, based on the mission results, the user with a first module on a second treatment pathway; and
providing missions to the client for the user to perform based on the first module on the second treatment pathway.

25. A treatment support method for providing personalized treatment support using predetermined treatment pathways, comprising:
positioning a patient at a first location, the first location being a first module on a first treatment pathway;
selecting first treatment support missions for the patient based on the first location;
providing at least one of the selected first treatment support missions to the patient;
receiving mission data for the at least one of the selected treatment support missions;
identifying, based on the received mission data, a secondary condition of the patient;
repositioning the patient at a second location, the second location being a first module on a second treatment pathway; and
providing at least one second treatment support mission for the patient based on the second location.

26. The treatment support method of claim 25, further comprising:
repositioning the patient at the first location, following completion of the at least one second treatment support mission; and
providing, after repositioning the patient at the first location, at least one third treatment support mission for the patient based on the first location.

27. A treatment support method, comprising:
selecting, based on a position of a user at a first module on a first predetermined treatment pathway, among first missions associated with the first module;
providing at least one of the selected first missions to a client for the user to perform;
obtaining, from the client, first mission data concerning performance of the at least one of the selected first missions;
updating the position of the user, based on the obtained first mission data, to a first module on a second predetermined treatment pathway;
selecting among second missions associated with the first module on the second predetermined treatment pathway; and
providing at least one of the selected second missions to the client.

28. The treatment support method of claim 27, wherein the method further comprises:
obtaining, from the client, second mission data concerning the at least one of the selected second missions;
determining that the user has satisfied a mastery condition;
updating the position of the user, based on the determination, to a second module on the second predetermined treatment pathway;
selecting among third missions associated with the second module on the second predetermined treatment pathway; and
providing at least one of the selected third missions to the client.

29. The treatment support method of claim 28, wherein:
the selected second missions include at least one mandatory mission; and
satisfaction of the mastery condition depends, at least in part, upon performance of the at least one mandatory mission.

30. The treatment support method of claim 27, wherein:
the second missions include a mission sequence; and
the selection of the second missions depends, in part, on a position of the user in the mission sequence.

31. The treatment support method of claim 27, wherein:
a value of a skill is associated with the user;
the second treatment pathway concerns development of the skill;
the method further comprises determining, based on the first received mission data, that the user requires development of the skill; and
the position of the user is updated to the first module on the second treatment pathway in response to the determination.

32. The treatment support method of claim 27, wherein:
the method further comprises:
reselecting among first missions associated with the first module on the first predetermined treatment pathway; and
providing, together with the at least one of the selected second missions, at least one of the reselected first missions.

33. The treatment support method of claim 32, wherein:
the first missions are each associated with a point value;
the first treatment pathway is associated with a point total; and
the reselection among the first missions is based on based on the point total and the point values.

34. The treatment support method of claim 27, wherein:
providing the at least one of the selected first missions comprises configuring the at least one of the selected first missions based on stored data concerning missions previously competed by the user.

35. The treatment support method of claim 27, wherein:
the selection of the second missions depends upon stored data concerning:
missions previously competed by the user; or
values of skills associated with the user.

36. The treatment support method of claim 27, wherein:
the operations further comprise:
receiving an identifier from the user; and
based on the identifier, associating the user with the first treatment path.

37. The treatment support method of claim 27, wherein:
the first mission data includes activity or biometric data acquired by a wearable device of the user.

38. A treatment support system, comprising:
at least one processor; and
at least one computer-readable medium containing instructions that, when executed by the at least one processor, cause the treatment support system to perform operations comprising:
provide a patient-specific list including a first interactive opportunity to a patient system associated with a patient;
obtain performance data from the patient system, the performance data concerning performance of the first interactive opportunity by the patient;
update a patient profile of the patient based on the obtained performance data;
generate a first competency-specific list by selecting among interactive opportunities according to a competency model and the patient profile;
generate a second patient-specific list of interactive opportunities by selecting among interactive opportunities included in the first competency-specific list and in at least one second competency-specific list according to a care model and the patient profile; and
provide the second patient-specific list to the patient system.

39. The treatment support system of claim 38, wherein:
the patient profile includes patient competency levels and corresponding target competency levels, and
the care model is configured to select among the interactive opportunities included in the first competency-specific list and in the at least one second competency-specific list based on differences between the target competency levels and the corresponding patient competency levels.

40. The treatment support system of claim 38, wherein:
the patient profile includes at least one patient competency level, and the care model is configured to select among the interactive opportunities based on the at least one patient competency level.

41. The treatment support system of claim 40, wherein:
the obtained performance data indicates successful completion of the first interactive opportunity; and
updating the patient profile based on the obtained performance data comprises increasing the at least one patient competency level.

42. The treatment support system of claim 38, wherein:
the patient profile includes an indication of a diagnosis of the patient; and
the care model is configured to select among the interactive opportunities included in the first competency-specific list and in the at least one second competency-specific list based on weights corresponding to the diagnosis.

43. The treatment support system of claim 38, wherein:
the patient profile includes patient interaction data; and
the competency model is configured to select among the interactive opportunities based on the patient interaction data.

44. The treatment support system of claim 38, wherein:
the care model selects interactive opportunities from the first competency-specific list and the at least one second competency-specific list according to constraints, the constraints including:
a number of interactive opportunities per day; and
satisfaction of a compatibility criterion for the selected interactive opportunities.

45. The treatment support system of claim 38, wherein:
the at least one second competency-specific list comprises multiple second competency-specific lists generated independently according to multiple second competency models and the patient profile.

46. The treatment support system of claim 38, wherein:
the first competency-specific list and the at least one second competency-specific list include duplicate interactive opportunities; and
generation of the second patient-specific list comprises deduplication of the duplicate interactive opportunities by the care model.

47. The treatment support system of claim 38, wherein:
the competency model corresponds at least one of a sleep management competency, stress management competency, activity level competency, diet or nutrition management competency, hydration management competency, weight-management competency, treatment-engagement competency, pain management competency, and mental state or psychological management competency.

48. The treatment support system of claim 38, wherein:
the performance data includes activity or biometric data acquired by a wearable device of the patient.

49. A treatment support system, comprising:
at least one processor; and
at least one computer-readable medium containing instructions that, when executed by the at least one processor, cause the treatment support system to perform operations comprising:
generating first lists by independently selecting interactive opportunities from a pool of interactive opportunities using first models, the first models selecting the interactive opportunities from the pool based on:
competency levels of a patient; and
associations between the interactive opportunities and competencies of the patient;
generating a second list by selecting interactive opportunities from the first lists using a second model, the second model selecting the interactive opportunities from the first lists based on:
the competency levels of the patient; and
target competency levels for the patient;
providing the second list to a client device of the patient;
receiving from the client device a selection of an interactive opportunity in the second list;
providing the selected interactive opportunity to the client device;
receiving performance data from the client device, the performance data concerning performance of the selected interactive opportunity by the patient; and
updating the competency levels of the patient based on the received performance data.

50. The treatment support system of claim 49, wherein:
the second model selects the interactive opportunities based on differences between the competency levels and corresponding ones of the target competency levels.

51. The treatment support system of claim 49, wherein:
the received performance data indicates successful completion of the selected interactive opportunity; and
updating a patient profile based on the received performance data comprises increasing values of competencies associated with the selected interactive opportunity.

52. The treatment support system of claim 51, wherein:
the first models select the interactive opportunities from the pool based in part on the patient profile.

53. The treatment support system of claim 51, wherein:
the first models select the interactive opportunities from the pool based in part on patient interaction data or patient information included in the patient profile.

54. The treatment support system of claim 49 wherein:
the second model selects interactive opportunities from the first lists according to constraints, the constraints including:
a number of interactive opportunities per day; and
satisfaction of a compatibility criterion for the selected interactive opportunities.

55. The treatment support system of claim 49, wherein:
the first lists include duplicate interactive opportunities; and
generation of the second list comprises deduplication of the duplicate interactive opportunities by the second model.

56. The treatment support system of claim 49, wherein:
the competencies of the patient include at least one of sleep management, stress management, activity level management, diet or nutrition management, hydration management, weight-management, treatment engagement, pain management, and mental state or psychological management.

57. The treatment support system of claim 49, wherein:
the received performance data includes activity or biometric data acquired by a wearable device of the patient.
